SEQUENCE LISTING (18 sequences)

SEQ ID NO: 1 - protein, 23 aa, Homo sapiens
SMEEAINERIQEVAGSLIFRAIS

SEQ ID NO: 2 - protein, 86 aa, Homo sapiens
SMEEAINERIQEVAGSLIFRAISSIGLECQSVTSRGDLATCPRGFAVTGCTCGSACGSWDVRAETTCHCQCAGMDWTGARCCRVQP

SEQ ID NO: 3 - protein, 1208 aa, Artificial Sequence
  MODIFIED SARS-COV-2 SPIKE ECTODOMAIN (Sm)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 4 - protein, 1208 aa, SARS-CoV-2 coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 5 - protein, 1298 aa, Artificial Sequence
  RESISTIN POLYPEPTIDE (T1) LINKED TO MODIFIED SPIKE ECTODOMAIN (Sm)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGGSMEEAINERIQEVAGSLIFRAISSIGLECQSVTSRGDLATCPRGFAVTGCTCGSACGSWDVRAETTCHCQCAGMDWTGARCCRVQP

SEQ ID NO: 6 - protein, 1314 aa, Artificial Sequence
  RESISTIN POLYPEPTIDE LINKED TO MODIFIED SPIKE ECTODOMAIN WITH C-TERMINAL FLAG AND 6xHIS TAGS (SmT1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGGSMEEAINERIQEVAGSLIFRAISSIGLECQSVTSRGDLATCPRGFAVTGCTCGSACGSWDVRAETTCHCQCAGMDWTGARCCRVQPDYKDDDDKGHHHHHHG

SEQ ID NO: 7 - DNA, 3897 bp, Artificial Sequence
  CODON-OPTIMIZED NUCLEOTIDE SEQUENCE ENCODING Sm
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtaccggcggttcaatggaggaggctatcaatgagcgtattcaggaagtcgcaggttcactgatttttcgggctatttcaagtatcggtctggagtgtcagtccgtgaccagcaggggcgacctggctacatgcccaaggggattcgctgtgaccggatgcacatgtggctctgcctgtggctcctgggacgtgagagctgagacaacatgccactgtcagtgcgctggaatggattggaccggagctcgctgctgtagggtccagcccgat

SEQ ID NO: 8 - DNA, 3945 bp, Artificial Sequence
  CODON-OPTIMIZED NUCLEOTIDE SEQUENCE ENCODING  S
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtaccggcggttcaatggaggaggctatcaatgagcgtattcaggaagtcgcaggttcactgatttttcgggctatttcaagtatcggtctggagtgtcagtccgtgaccagcaggggcgacctggctacatgcccaaggggattcgctgtgaccggatgcacatgtggctctgcctgtggctcctgggacgtgagagctgagacaacatgccactgtcagtgcgctggaatggattggaccggagctcgctgctgtagggtccagcccgattacaaagacgacgacgataaaggtcaccatcaccaccatcacggttaa

SEQ ID NO: 9 - protein, 27 aa, T4 phage
GYIPEAPRDGQAYVRKDGEWVLLSTFL

SEQ ID NO: 10 - protein, 32 aa, Saccharomyces cerevisiae
MKQIEDKIEEILSKIYHIENEIARIKKLIGEV

SEQ ID NO: 11 - protein, 1314 aa, Artificial Sequence
  RESISTIN POLYPEPTIDE LINKED TO WILD-TYPE SPIKE ECTODOMAIN WITH C-TERMINAL 6xHIS TAG (ST1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGGSMEEAINERIQEVAGSLIFRAISSIGLECQSVTSRGDLATCPRGFAVTGCTCGSACGSWDVRAETTCHCQCAGMDWTGARCCRVQPDYKDDDDKGHHHHHHG

SEQ ID NO: 12 - protein, 1285 aa, Artificial Sequence
  T4 FOLDON TRIMERIZATION DOMAIN  LINKED TO WILD- TYPE SPIKE ECTODOMAIN WITH FLAG tag, Dual Streptag, and C-TERMINAL 6xHIS TAG
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGSSGGYIPEAPRDGQAYVRKDGEWVLLSTFLTGGDYKDDDDKGWSHPQFEKGGGSGGGSWSHPQFEKGHHHHHHG

SEQ ID NO: 13 - protein, 1265 aa, Artificial Sequence
  GCN4 TRIMERIZATION DOMAIN  LINKED TO WILD-TYPE SPIKE ECTODOMAIN WITH C-TERMINAL FLAG AND 6xHIS TAGS (ST3)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGSSGMKQIEDKIEEILSKIYHIENEIARIKKLIGEVTGGDYKDDDDKGHHHHHHG

SEQ ID NO: 14 - protein, 1285 aa, Artificial Sequence
  T4 FOLDON TRIMERIZATION DOMAIN  LINKED TO MODIFIED SPIKE ECTODOMAIN WITH FLAG TAG, Dual Streptag, and C-TERMINAL 6xHIS TAG (SmT2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGSSGGYIPEAPRDGQAYVRKDGEWVLLSTFLTGGDYKDDDDKGWSHPQFEKGGGSGGGSWSHPQFEKGHHHHHHG

SEQ ID NO: 15 - protein, 1265 aa, Artificial Sequence
  GCN4 TRIMERIZATION DOMAIN  LINKED TO MODIFIED SPIKE ECTODOMAIN WITH C-TERMINAL FLAG AND 6xHIS TAGS (SmT3)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGSSGMKQIEDKIEEILSKIYHIENEIARIKKLIGEVTGGDYKDDDDKGHHHHHHG

SEQ ID NO: 16 - protein, 1208 aa, Artificial Sequence
  MODIFIED SARS-COV-2 SPIKE ECTODOMAIN 2 (Sm2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 17 - protein, 1314 aa, Artificial Sequence
  RESISTIN POLYPEPTIDE LINKED TO MODIFIED SPIKE ECTODOMAIN 2 WITH C-TERMINAL FLAG AND 6xHIS TAGS (Sm2T1)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTGGSMEEAINERIQEVAGSLIFRAISSIGLECQSVTSRGDLATCPRGFAVTGCTCGSACGSWDVRAETTCHCQCAGMDWTGARCCRVQPDYKDDDDKGHHHHHHG

SEQ ID NO: 18 - DNA, 3626 bp, Artificial Sequence
  CODON OPTIMIZED NUCLEOTIDE SEQUENCE ENCODING Sm2
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacaaggtggaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcaggg

(19) United States
(12) Patent Application Publication
(10) Pub. No.: US 2023/0174591 A1
(43) Pub. Date: Jun. 8, 2023

(54) Use of Human Resistin as a Trimerization Partner for Expression of Trimeric Proteins

(71) Applicant: National Research Council of Canada, Ottawa (CA)

(72) Inventor: Yves DUROCHER, Montreal (CA)

(73) Assignee: National Research Council of Canada, Ottawa, ON (CA)

(21) Appl. No.: 17/999,071

(22) PCT Filed: May 18, 2021
(86) PCT No.: PCT/IB2021/054285

§ 371 Date: Nov. 17, 2022

Related U.S. Application Data
(60) Provisional application No. 63/027,109, filed on May 19, 2020.

(51) Int. Cl. C07K 14/005; G01N 33/569; G01N 33/543; C07K 14/575; A61K 39/215

(52) U.S. Cl. CPC C07K 14/005; G01N 33/56983; G01N 33/54386; C07K 14/575; A61K 39/215; G01N 2333/165; G01N 2469/20; C07K 2319/00; C12N 2770/20022; C12N 2770/20034; C12N 2770/20071

(57) ABSTRACT

Provided is a recombinant polypeptide comprising a resistin trimerization domain and a polypeptide of interest. Further provided is an expression vector encoding the recombinant polypeptide and a method of expressing the recombinant polypeptide. The polypeptide of interest may be a trimeric viral surface antigen or a portion thereof, such as the ectodomain of the SARS-CoV-2 spike protein. Further provided are compositions, such as immunogenic compositions and vaccines, comprising the recombinant polypeptide.

### CROSS-REFERENCE TO RELATED APPLICATIONS

This application claims the benefit of priority of U.S. provisional patent application No. 63/027,109, filed May 19, 2020, the content of which is hereby incorporated by reference in its entirety.

### FIELD

The invention relates to the field of recombinant protein production, particularly to the recombinant production of protein trimers such as trimeric viral surface antigens, and more particularly to the production of coronavirus spike protein, particularly SARS-CoV-2 spike protein.

### BACKGROUND

Many enveloped viruses, such as influenza, HIV, RSV, hMPV and others, express trimeric surface antigens that are critical for their infectivity. For example, influenza expresses the hemagglutinin protein, a transmembrane homotrimer that binds sialic acid sugar residues exposed on cell surface glycoproteins. Respiratory syncytial virus (RSV) expresses the RSV-F glycoprotein, a transmembrane homotrimeric protein that binds a yet undefined receptor. Similarly, the SARS-CoV-2 spike (S) protein is a trimeric transmembrane protein that binds the ACE2 receptor.

Trimerization domains can be used to promote trimerization of proteins, including soluble viral proteins. The most widely known and used trimerization domains are the T4 phage fibritin trimerization domain (foldon) (Tao et al. 1997) and the yeast GCN4 trimerization domain (Harbury et al. 1993). A problem with trimerization domains that are commonly used to develop trimeric viral antigen subunit vaccines is their potential immunogenicity in humans (Sliepen et al. 2015).

The SARS-CoV-2 spike protein is closely related to the spike protein of SARS-CoV-1 (also referred to as SARS-CoV), the virus responsible for the outbreak of severe acute respiratory syndrome (SARS) that occurred in 2003. SARS-CoV-2 is the virus responsible for the COVID-19 pandemic that started in late 2019. Both proteins are large, multi-domain glycoproteins with transmembrane domains that traverse the viral envelope and that are proteolytically processed into S1 and S2 subunits. Notably, while the SARS-CoV-1 spike protein is only cleaved during infection of target cells, the SARS-CoV-2 spike protein contains a furin recognition site at the S1/S2 junction, such that cleavage occurs during biosynthesis in host cells; this difference may impact the route of entry of the two virus types into host cells (Xia et al, 2020). The SARS-CoV-1 spike protein was shown to assemble into homo-trimeric complexes that are found on mature viral particles (Gui et al, 2017).

In the literature on SARS-CoV-1 and other related coronaviruses, there are reports of various approaches for producing recombinant spike proteins. Individual domains of the spike polypeptide, including the receptor-binding and hemagglutinin-esterase domains, have been produced in CHO, HEK293, Vero and insect cells (Li et al, 2013; Du et al, 2010; Huang et al, 2015). It is also possible to express the full-length spike polypeptide, including transmembrane and C-terminal domains, which can be purified following membrane solubilisation of expressing cells (Kam et al, 2007; Coleman et al, 2014). Finally, expression of full-length soluble forms of coronavirus spike ectodomains has also been reported in HEK293 and insect cells (Li et al, 2013; Tortorici et al, 2019; Chun et al, 2019; Kirchdoerfer et al, 2018; Wrapp et al, 2020; Walls et al, 2020). Importantly, however, in cases where this data is reported, yields were extremely low, ranging from 0.5-1.5 mg per litre of culture media for expression of constructs containing the full-length spike ectodomain (Kam et al, 2007; Wrapp et al, 2020). This productivity is well below desirable levels for mass production, in particular for development and manufacturing for potential diagnostic or vaccine applications.

### SUMMARY

The present inventors have found that resistin is an effective trimerization partner for the recombinant production of trimeric proteins, including trimeric viral surface antigens such as the ectodomain of the SARS-CoV-2 spike protein.

Accordingly, there is provided a recombinant polypeptide comprising a polypeptide of interest and a resistin trimerization partner, wherein the trimerization partner advantageously facilitates the trimerization of the polypeptide of interest. In an embodiment, the protein of interest is a SARS-CoV-2 spike protein ectodomain and the recombinant polypeptide provides a properly folded and assembled SARS-CoV-2 antigen that is structurally equivalent to the native antigen. In an embodiment, the trimerization partner is a resistin polypeptide, a polypeptide comprising the amino acid sequence set forth in SEQ ID NO: 1, or a sequence substantially equivalent thereto.

An embodiment of the present disclosure is a recombinant polypeptide comprising a first portion and a second portion, the first portion comprising a resistin trimerization domain and the second portion comprising a viral surface antigen or a fragment thereof. In an embodiment, the first portion comprises an amino acid sequence having at least 85% sequence identity to the full length of the amino acid sequence set forth in SEQ ID NO: 1 and the second portion comprises an amino acid sequence of a viral surface antigen. In an embodiment, the first portion comprises an amino acid sequence having at least 85%, at least 90%, or at least 95% sequence identity to the full length of the amino acid sequence set forth in SEQ ID NO: 1. In an embodiment, the first portion comprises or consists of the amino acid sequence set forth in SEQ ID NO: 1.

In an embodiment, the first portion is located C-terminal of the second portion.

In an embodiment, the first portion is linked to the second portion by a linker.

In an embodiment, the first portion comprises an amino acid sequence having at least 85% identity to the full length of the amino acid sequence set forth in SEQ ID NO: 2. In an embodiment, the first portion comprises an amino acid sequence having at least 85%, at least 90%, at least 92%, at least 94%, at least 96%, or at least 98% identity to the full length of the amino acid sequence set forth in SEQ ID NO: 2. In an embodiment, the second portion comprises or consists of the amino acid sequence set forth in SEQ ID NO: 2.

In an embodiment, the second portion comprises an amino acid sequence having at least 70% identity to the full length of the amino acid sequence set forth in SEQ ID NO: 3, SEQ ID NO: 4, or SEQ ID NO: 16. In an embodiment, the second portion comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, or at least 99% identity to the full length of the amino acid sequence set forth in SEQ ID NO: 3, SEQ ID NO: 4, or SEQ ID NO: 16. In an embodiment, the second portion comprises or consists of the amino acid sequence set forth in SEQ ID NO: 3, SEQ ID NO: 4, or SEQ ID NO: 16.

In an embodiment, the recombinant polypeptide comprises an amino acid sequence having a least 85% identity to the full length of the amino acid sequence set forth in SEQ ID NO: 5, SEQ ID NO: 6, or SEQ ID NO: 17. In an embodiment, the recombinant polypeptide comprises an amino acid sequence having at least 85%, at least 90%, at least 91%, at least 92%, at least 93%, at least 94%, at least 95%, at least 96%, at least 97%, at least 98%, or at least 99% identity to the full length of the amino acid sequence set forth in SEQ ID NO: 5, SEQ ID NO: 6, or SEQ ID NO: 17. In an embodiment, the recombinant polypeptide comprises or consists of the amino acid sequence set forth in SEQ ID NO: 5, SEQ ID NO: 6, or SEQ ID NO: 17.

In an embodiment, the recombinant polypeptide further comprises at least one affinity tag. In an embodiment, the at least one affinity tag comprises a FLAG tag and/or a 6×His tag. In an embodiment, the at least one affinity tag is located C-terminal of the first and second portions.

Another embodiment is a trimeric protein comprising three recombinant polypeptides as described herein. In an embodiment, each of the three recombinant polypeptides comprises the same amino acid sequence. In an embodiment, each of the three recombinant polypeptides consists of the same amino acid sequence.

Another embodiment is a composition comprising a recombinant polypeptide or a trimeric protein as described herein and a pharmaceutically acceptable carrier.

In an embodiment, the composition further comprises an adjuvant. In an embodiment, the adjuvant comprises 6′-sulfate-β-D-Galp-(1,4)-β-D-Glcp-(1,1)-archaeol (SLA), monophosphoryl lipid A (MPL), aluminum phosphate, QS-21, and/or liposomes. In an embodiment, the adjuvant comprises MPL, QS-21, and liposomes. In an embodiment, the adjuvant comprises SLA. In an embodiment, the adjuvant comprises aluminum phosphate. In an embodiment the adjuvant comprises CpG oligodeoxynucleotide (CpG) or polyinosinic-polycytidylic acid (Poly(I:C)). In an embodiment the adjuvant comprises CpG or Poly(I:C) in combination with SLA.

In an embodiment, the composition is an immunogenic composition.

In an embodiment, the composition is a vaccine.

Another embodiment is an expression vector for producing a recombinant polypeptide as described herein, the expression vector comprising a nucleic acid molecule, the nucleic acid molecule encoding a recombinant polypeptide as described herein, operably linked to one or more regulatory elements that allow the nucleic acid molecule to be expressed. In an embodiment, the nucleic acid molecule is operably linked to a promoter. In an embodiment, the promoter is an inducible promoter. In an embodiment, the promoter is a cumate-inducible promoter.

In an embodiment, the nucleic acid molecule comprises the nucleotide sequence set forth in SEQ ID NO: 7, SEQ ID NO: 8, or SEQ ID NO: 18.

Another embodiment is a method for expressing a recombinant polypeptide as described herein, the method comprising: introducing an expression vector as described herein into a host cell and maintaining the cell under conditions that allow the recombinant polypeptide to be expressed. In an embodiment, the host cell is a mammalian cell. In an embodiment, the host cell is a Chinese Hamster Ovary (CHO) cell.

Another embodiment is use of a recombinant polypeptide or trimeric protein as described herein as an antigen in a vaccine, immunogenic composition, or serological assay.

Another embodiment is a method of identifying whether a subject has developed an antibody response to a pathogen or to an antigen from a pathogen, the method comprising
a) contacting serum or a serum fraction from the subject with a recombinant polypeptide as described herein, and
b) detecting binding of the recombinant polypeptide by an antibody present in the serum or the serum fraction,
   wherein the second portion of the recombinant polypeptide comprises an amino acid sequence from an antigen of the pathogen.

In an embodiment, the pathogen is SARS-CoV-2 and the recombinant polypeptide comprises a SARS-CoV-2 spike protein ectodomain sequence as described herein.

In an embodiment, a recombinant polypeptide or trimeric protein as described herein is immobilized on a surface.

A further embodiment is a device comprising a recombinant polypeptide or a trimeric protein as described herein immobilized on a surface. In an embodiment, the device is a plate or a test cassette.

A further embodiment is a kit comprising a recombinant polypeptide or a trimeric protein as defined herein, and an antigen-binding molecule that specifically binds the recombinant polypeptide or trimeric protein. In an embodiment, the antigen-binding molecule is an antibody or an antigen-binding fragment thereof. In an embodiment, the antigen-binding molecule is bonded to a detectable label.

### BRIEF DESCRIPTION OF DRAWINGS

FIG. 1 shows the three dimensional structure of a human resistin trimer, left, and a trimer of a recombinant polypeptide according to the present disclosure comprising a stabilized SARS-CoV-2 spike ectodomain polypeptide and a human resistin polypeptide (SmT1), right.

FIG. 2 shows a schematic of a recombinant SARS-CoV-2 spike ectodomain-resistin fusion polypeptide, with the N-terminal end of the polypeptide to the left and the C-terminal end of the polypeptide to the right. “S1” and “S2” represent the S1 and S2 subunits of the spike protein.

FIG. 3 shows the sequence of the SmT1 recombinant polypeptide. The modified SARS-CoV-2 spike sequence is shown in unboxed plain text, the linker sequence is shown in bold, the human resistin sequence is boxed by a solid line and the putative trimerization domain of the human resistin sequence is underlined, the FLAG tag sequence is boxed by a dashed line, and the 6×His tag sequence is boxed by a double solid line.

FIG. 4 shows western blots (upper panels) of culture supernatants taken at 5 or 6 days post-transfection from HEK293-6E (Stuible et al, 2018) or CHO-3E7 (L′Abbé et al, 2018) cells expressing recombinant polypeptides comprising: the full ectodomain of the SARS-CoV-2 spike protein (aa 1-1028 of GenBank protein ID QHD43416.1), a human resistin sequence (aa 23 to 108 of GenBank accession NM 001193374.2) joined to the C-terminus of the ectodomain by a peptide linker, and C-terminal FLAG and 6×His affinity tags. Two variants of the SARS-CoV-2 ectodomain were used, an unmodified ectodomain sequence (S) and a modified ectodomain sequence (Sm) that includes two modifications, one to prevent furin-mediated S1/S2 cleavage (RRAR->GGAS) and the second to stabilize the pre-fusion conformation (K986P V987P), as recently described in Wrapp et al, 2020. The fusion proteins including the resistin sequence are referred to as ST1 (wild-type S ectodomain protein sequence) and SmT1 (modified S ectodomain protein sequence). The lower panels show Ponceau S staining of the membranes before western blotting.

FIG. 5 shows a Coomassie®-stained 4-12% SDS-PAGE gel in which 2 μg of purified S, Sm, ST1, and SmT1 proteins were separated under reducing conditions.

FIG. 6 provides the elution profiles of wild-type spike protein (S), upper panel, and a mutated spike ectodomain (Sm), lower panel, on a Superose® 6 5/15 column equilibrated in PBS.

FIG. 7 provides the elution profiles of wild-type spike ectodomain-resistin fusion (ST1), upper panel, and the mutated spike ectodomain-resistin fusion (SmT1), lower panel, on a BEH SEC200 UPLC column equilibrated in PBS.

FIG. 8 provides the SEC-UPLC elution profiles of mutated spike ectodomain (Sm) fused to three different trimerization partners T1=resistin, T2=T4Fib, T3=GCN4.

FIG. 9 Affinity purified S proteins were analyzed by reducing SDS-PAGE and stained by Coomassie® blue. Molecular weight protein standards are shown on the last lane on the right. Note that ST2 construct lost significant amount of the cleaved S1 fragment while ST3 the loss is almost complete.

FIG. 10 shows an alignment of amino acids 661 to 1020 of modified spike ectodomain (Sm, SEQ ID NO: 3), modified spike ectodomain 2 (Sm2, SEQ ID NO: 16), and wild-type spike ectodomain (S, SEQ ID NO: 4), showing the locations of: the RRAR->GGAS mutations in Sm and Sm2, and the K986P and V987P mutations in Sm.

FIG. 11 shows the sequence of the Sm2T1 recombinant polypeptide. The modified SARS-CoV-2 spike sequence is shown in unboxed plain text, the linker sequence is shown in bold, the human resistin sequence is boxed by a solid line and the putative trimerization domain of the human resistin sequence is underlined, the FLAG tag sequence is boxed by a dashed line, and the 6×His tag sequence is boxed by a double solid line.

FIG. 12 shows IgG titers in serum from male and female mice immunized with a single dose of the SmT1 vaccine formulations identified in Table 1.

FIG. 13 shows IgG titers in serum from male and female mice immunized with two doses of the SmT1 vaccine formulations identified in Table 1.

FIG. 14 shows IgG1 and IgG2 profiles in serum of male and female mice immunized with two doses of the SmT1 vaccine formulations identified in Table 1.

FIG. 15 shows the results of IFN-γ ELISpot assays using N-terminal and C-terminal peptide pools, carried out on splenocytes from male and female mice immunized with two doses of the SmT1 vaccine formulations identified in Table 1.

FIG. 16 shows the results of IFN-γ ELISpot assays using whole spike protein, carried out on splenocytes male and female mice immunized with two doses of the SmT1 vaccine formulations identified in Table 1.

FIG. 17 shows the results of intracellular cytokine staining (ICCS) on splenocytes from female mice immunized with two doses of the SmT1 vaccine formulations identified in Table 1. Graphs indicate the number of Ag-specific IFN-γ, IL-2 or TNF-α-positive cells per million CD4 T cells following stimulation of splenocytes with N-terminal and C-terminal peptide pools or whole spike protein.

FIG. 18 shows the results of intracellular cytokine staining (ICCS) on splenocytes from female mice immunized with two doses of the SmT1 vaccine formulations identified in Table 1. Graphs indicate the number of Ag-specific IFN-γ, IL-2 or TNF-α-positive cells per million CD8 T cells following stimulation of splenocytes with N-terminal and C-terminal peptide pools or whole spike protein.

FIG. 19 shows IgG titers in serum from female mice immunized with a single dose of the SmT1 vaccine formulations identified in Table 2 (Groups 1-11).

FIG. 20 shows the results of IFN-γ ELISpot assays using N-terminal and C-terminal peptide pools, carried out on splenocytes of female mice immunized with two doses of the SmT1 vaccine formulations identified in Table 2 (Groups 1-11).

FIG. 21 shows the neutralization activity on the binding of soluble spike protein to VERO cells of serum from mice immunized with two doses of the SmT1 vaccine formulations identified in Table 2 (Groups 1-11).

FIG. 22 shows IgG titers in serum from female mice immunized with a single dose of the SmT1 or Sm2T1 vaccine formulations identified in Table 2 (Groups 1, 2, 4, 12-16).

FIG. 23 shows the results of IFN-γ ELISpot assays using N-terminal and C-terminal peptide pools, carried out on splenocytes of female mice immunized with two doses of the SmT1 vaccine formulations identified in Table 2 (Groups 1, 2, 4, 12-16).

FIG. 24 shows the neutralization activity on the binding of soluble spike protein to VERO cells of serum from mice immunized with two doses of the SmT1 vaccine formulations identified in Table 2 (Groups 1, 2, 4, 12-16).

FIG. 25 shows the degree of body weight loss following SARS-CoV-2 challenge in female hamsters immunized with the SmT1 vaccine formulations identified in Table 3.

FIG. 26 shows the viral load in lungs following SARS-CoV-2 challenge in female hamsters immunized with the SmT1 vaccine formulations identified in Table 3.

FIG. 27 shows IgG titers in serum from female hamsters immunized with the SmT1 vaccine formulations identified in Table 3.

FIG. 28 shows the neutralization activity on the binding of soluble spike protein to VERO cells of serum from hamsters immunized with the SmT1 vaccine formulations identified in Table 3.

FIG. 29 shows an SDS-PAGE gel of a purified VHH-resistin fusion protein.

FIG. 30 shows a SEC-UPLC profile of the purified VHH-resistin fusion protein.

FIG. 31 shows an SDS-PAGE gel of purified Sm2T1 fusion protein. Lane 1: MW standards; lane 2: clarified harvest (expression level estimated at 100 mg/L); lane3: IMAC flow-through; lane 4-6 IMAC washes; lane 7-10: IMAC elution; lane 12: pooled IMAC eluted fractions; lane 13; 3 μg of purified Sm2T1 was loaded on the gel to show purity.

FIG. 32 shows a SEC-UPLC profile of purified Sm2T1 fusion protein

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures, published sequences, and other references mentioned herein are expressly incorporated by reference in their entirety.

### Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

The term “about” as used herein may be used to take into account experimental error, measurement error, and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence).

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, modified as in Karlin and Altschul, 1993. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present invention. To obtain gapped alignments for comparison purposes, Gapped BLAST can be utilized as described in Altschul et al., 1997. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

A “substantially identical” sequence may comprise one or more conservative amino acid mutations. It is known in the art that one or more conservative amino acid mutations to a reference sequence may yield a mutant peptide with no substantial change in physiological, chemical, physico-chemical or functional properties compared to the reference sequence; in such a case, the reference and mutant sequences would be considered “substantially identical” polypeptides. A conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pKa value of greater than 7, which are typically positively charged at physiological pH. Basic amino acids include arginine (Arg or R) and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of (Eisenberg et al, 1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pKa value of less than 7, which are typically negatively charged at physiological pH. Acidic amino acids include glutamate (Glu or E), and aspartate (Asp or D). Histidine (His or H) is a polar amino acid with a special ionization potential due to its pKa around 7, and more precisely around 6.4 in case of histidine residues located at the protein surface (Tanokura, 1983). This results in histidine amino acid residues being a “polar” and predominantly uncharged at physiological pH of 7.2-7.4, and predominantly positively charged in acidic environments (pH<7).

The substantially identical sequences of the present invention may be at least 85% identical; in another example, the substantially identical sequences may be at least 85%, at least 90%, at least 91%, at least 92%, at least 93%, at least 94%, at least 95%, at least 96%, at least 97%, at least 98%, at least 99%, or 100% identical, or any percentage there between, at the amino acid level or the nucleotide level to sequences described herein. Importantly, the substantially identical sequences retain the activity and specificity of the reference sequence. In a non-limiting embodiment, the difference in sequence identity may be due to conservative amino acid mutation(s). In a non-limiting embodiment, the difference in sequence identity may be due to synonymous nucleotide substitutions or nucleotide substitutions that give rise to conservative amino acid mutation(s). In a non-limiting example, the present invention may be directed to trimerization partner comprising an amino acid sequence that is at least 85%, at least 90%, or at least 95% identical to the trimerization domain sequence set forth in SEQ ID NO: 1.

As used herein the terms “peptide” and “polypeptide” refer to a linear chain of two or more amino acids joined by peptide bonds. The term “peptide” is generally used to refer to a short chain of amino acids comprising 2 to 49 amino acids, whereas the term “polypeptide” is generally used to refer to a longer chain of amino acids comprising 50 or more amino acids. However, these terms may be used interchangeably. The term “protein” is used herein to refer to one or more peptides or polypeptides that have been folded and/or assembled to form a three dimensional structure, although the terms protein and polypeptide may also be used interchangeably. A protein may include post-translational modifications, as will be understood to one skilled in the art. For example, a protein may be glycosylated, lipidated, phosphorylated, ubiquitinated, acetylated, nitrosylated, and/or methylated.

As used herein, the term “recombinant polypeptide” refers to a polypeptide that is produced by recombinant techniques, wherein generally DNA or RNA encoding the expressed protein is inserted into a suitable expression vector that is in turn introduced into a host cell to allow expression of the recombinant polypeptide. Recombinant polypeptides may include amino acid sequences from two or more sources, such as different proteins. Such recombinant polypeptides may be referred to as fusion polypeptides, fusion proteins, or fusion constructs. Recombinant polypeptides may also include one or more synthetic amino acid sequences.

As used herein, the term “linker” refers to a molecule that covalently links two polypeptides. The linker may be an amino acid, or a peptide comprising two or more amino acids. If the linker is an amino acid or peptide, the N-terminal end of the linker may be covalently linked by a peptide bond to the C-terminal end of a first polypeptide and the C-terminal end of the linker may be covalently linked by a peptide bond to the N-terminal end of a second polypeptide. Typically, the two polypeptides covalently linked by the linker are polypeptides that are not naturally joined, for example they may be encoded by different genes and/or by different species, or they may be different portions or domains of a single polypeptide or protein.

As used herein, the term “trimerization partner” refers to a trimerization domain or motif, or a polypeptide comprising a trimerization domain or motif, that is able to form a trimer. A trimerization partner may be included in a recombinant polypeptide to promote trimerization of the recombinant polypeptide to form a trimeric protein. In an embodiment the trimerization partner is a resistin polypeptide or the trimerization partner comprises a trimerization domain of a resistin polypeptide. In a preferred embodiment, the trimerization partner is a human resistin polypeptide or the trimerization partner comprises a trimerization domain of a human resistin polypeptide.

As used herein, the term “antigen” refers to any molecule, moiety or entity that is capable of eliciting an immune response. This includes cellular and/or humoral immune responses. An antigen is commonly a biological molecule, usually a protein, peptide, polysaccharide, lipid and/or conjugate that contains at least one epitope to which a cognate antibody can selectively bind.

As used herein, the term “antigen from a pathogen” refers to an antigen that is comprised by a pathogen, such as a virus, bacterium, or fungus, or that is derived from a pathogen. An antigen derived from a pathogen may, for example, be isolated from the pathogen, or the antigen may be engineered to replicate an antigen comprised by a pathogen. For example an antigen derived from a pathogen may be produced synthetically, semi-synthetically, or recombinantly. An antigen may be produced recombinantly; for example, by introducing a nucleic acid molecule encoding the antigen into a host cell or organism that is able to transcribe and/or translate the nucleic acid molecule to produce the antigen. In an embodiment, the pathogen is a pathogen that infects one or more animals, including human and non-human animals. In an embodiment, the pathogen is a pathogen that infects one or more mammals. In a specific embodiment, the pathogen is a virus that infects humans.

A “viral surface antigen” is an antigen, such as a polypeptide, that can be found on the surface of a virus. The viral surface antigen may be a trimeric viral surface antigen. Examples of trimeric viral surface antigens include but are not limited to Influenza hemagglutinin (HA), human immunodeficiency virus (HIV) gp120, Respiratory syncytial virus (RSV) RSVF protein, the Rabies Virus Glycoprotein (RABVG), and the Human metapneumovirus (hMPV) glycoprotein.

As used herein, the term “immunogenic composition” refers to any composition comprising an antigen that can be used to elicit an immune response in a subject. In specific embodiments, an immunogenic composition may further comprise an adjuvant.

As used herein, the term “pharmaceutically acceptable carrier” refers to a carrier that is non-toxic. Suitable pharmaceutically acceptable carriers include, for example, one or more of water, saline, phosphate buffered saline, dextrose, glycerol, ethanol, and combinations thereof. Pharmaceutically acceptable carriers may further contain minor amounts of auxiliary substances such as wetting or emulsifying agents, preservatives or buffering agents that enhance shelf life or effectiveness.

As used herein, the term “serological assay” refers to a diagnostic assay that relies on binding of an antibody to an antigen. A serological assay may also be referred to as a “serologic test”, “serological test”, or “serology test”. Serological assays may be used to detect the presence of antibodies against an antigen of interest in the serum of a subject. A recombinant polypeptide or protein as described herein may be used as the antigen in a serological assay. For example, a recombinant polypeptide comprising a SARS-CoV-2 spike protein ectodomain may be used as an antigen in a serological assay to identify subjects that have been infected with the SARS-SoV-2 virus. Examples of serology assays are flocculation tests, enzyme-linked immunosorbent assays (ELISAs), and western blots.

As used herein, the term “fragment”, in reference to a molecule, such as a nucleic acid molecule or a polypeptide, refers to a portion of the molecule that is less than the full length of the molecule.

As used herein, the term “subject” refers to a human or non-human animal, for example a mammal, avian, reptile, fish, or amphibian.

As used herein, the term “antigen-binding molecule” refers to a molecule that is able to specifically bind to an antigen, such as a recombinant polypeptide or trimeric protein as described herein. An “antigen-binding molecule” may, but need not be, an antibody or an antigen-binding fragment of an antibody. An antigen-binding fragment of an antibody (also referred to as an antigen-binding antibody fragment) is any antibody fragment that has the ability to specifically bind an antigen or epitope. Examples of antigen-binding antibody fragments include, but are not limited to, antigen-binding fragments (Fabs), F(ab′)2 fragments, Fab′ fragments, Fv fragments, single chain variable fragments (scFv), nanobodies (also known as V_{H}Hs), V_{H} fragments, and V_{L} fragments.

The specificity of an antigen-binding molecule, also referred to as “specific binding” or “specifically binding” or other grammatical forms thereof, can be determined based on affinity. A specific antigen-binding molecule preferably has a binding affinity (Kd) for its epitope of less than 10⁻⁷ M, preferably less than 10⁻⁸ M. An antigen-binding molecule may cross-react with more than one epitope or antigen and still be considered specific. For example, an antigen-binding molecule specific for SARS-CoV-2 spike protein may also specifically bind to variants of that spike protein and/or it may also specifically bind to spike proteins from one or more additional coronaviruses.

As used herein, the term “detectable label” refers to a molecule that can be bonded to a molecule of interest to allow the molecule of interest to be detected. Detectable labels are most commonly bonded by covalent bonding, but non-covalent bonding is also possible. Detection of the detectable reagent may be by direct observation (e.g. by microscopy or radiography), or by indirect observation (e.g. by exposing the detectable label to one or more reagents to allow a reaction between the detectable label and the one or more reagents to produce a detectable signal). Suitable detectable labels will be known to one skilled in the art and include, for example: radioisotopes; enzymes, such as horse radish peroxidase (HRP), calf intestinal alkaline phosphate (AP), glucose oxidase, and β-galactosidase; fluorophores; biotin; and colloidal gold. As used herein, the term detectable label includes molecules that are not typically detected directly, but that can be specifically bound by another detectable molecule. For example, a primary antibody or antibody fragment may be considered to be a detectable label, even if its detection involves the use of a secondary antibody labeled with a detectable label.

It should also be understood that, in certain methods described herein that include more than one step or act, the order of the steps or acts of the method is not necessarily limited to the order in which the steps or acts of the method are recited unless the context indicates otherwise.

### Description

To address problems of immunogenicity, misfolding, and lack of stability found with commonly used trimerization domains, the present inventors have discovered that a resistin protein or trimerization domain may be used as a trimerization partner for the production of recombinant polypeptides, such as viral surface antigens, and particularly trimeric viral surface antigens such as the SARS-CoV-2 spike protein ectodomain.

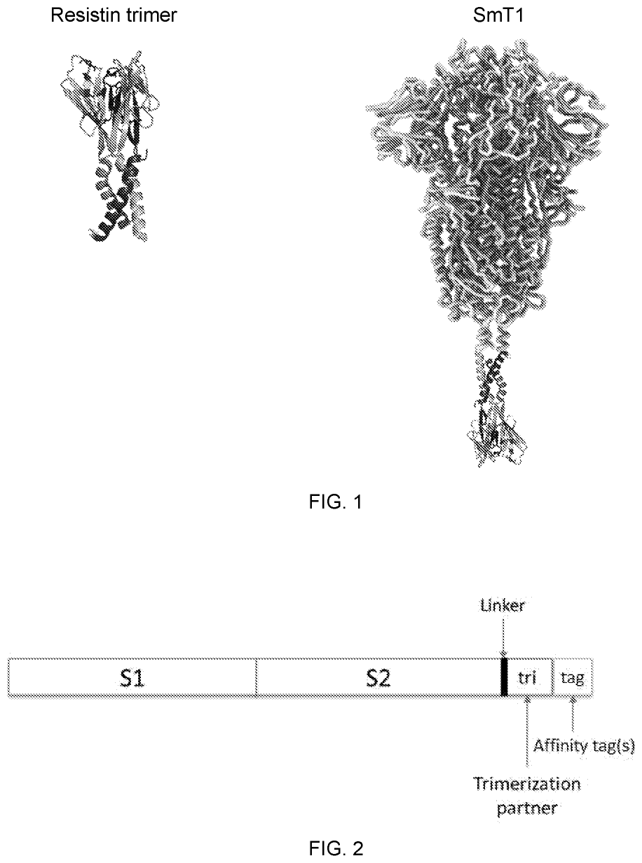

Resistin is a small secretory protein predominantly produced by macrophages that has an extremely stable and high-order multimeric structure, plays a role in inflammation, and also functions as a small accessory chaperone. Resistin exists in circulation mostly as trimeric and hexameric species, the hexameric form being formed through head-to-head covalent association of two resistin trimers by disulfide bridge formation via their penultimate N-terminal cysteine residues. The three-dimensional structure of the human resistin trimer is shown in FIG. 1. Since human resistin is naturally present in human serum, it is expected to be non-immunogenic or of low immunogenicity in humans. However, it is also possible to use a resistin protein or resistin trimerization domain from a non-human species, such as, but not limited to, a mammalian species, for example mouse or rat.

Resistin is involved in secretion of immune effectors and induces macrophages, PBMCs and hepatic stellar cells to secrete tumor necrosis factor alpha, interleukin (IL)-1b, IL-6, IL-8, IL-12 and MCP-1 that stimulate pro-inflammatory responses. As such, circulatory resistin levels have been positively correlated with common inflammatory conditions in humans. In contrast, resistin attenuates the inflammatory and immunological pathways of other types of immune cells. In dendritic cells, resistin suppresses the pro-inflammatory pathway and impairs antigen uptake. Resistin also diminishes the T-cell-mediated immune response by altering IRF-1-mediated TREG pathway.

The adenylate cyclase-associated protein-1 (CAP-1) has been identified as a receptor for resistin. Resistin stimulation of the CAP-1 receptor participates in resistin-mediated pro-inflammatory responses.

Resistin is highly stable and resistant to heat and chemical denaturants such as urea and SDS. It also protects other proteins from thermal aggregation, restores their functional activity after guanidinium chloride-induced denaturation and rescues bacteria from heat shock, suggesting that resistin has chaperone-like activity. This is also supported by the fact that tunicamycin/thapsigargin-induced ER stress and apoptosis are averted by the overexpression of recombinant resistin in HeLa and U937 cells.

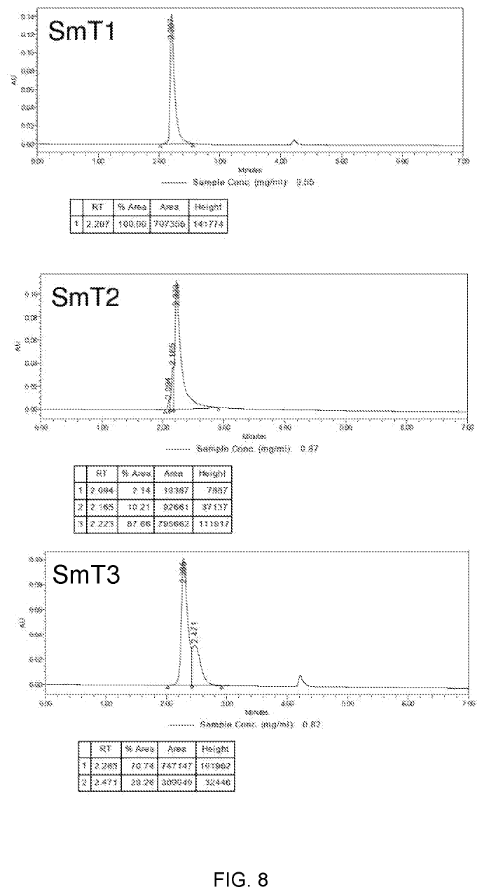

The present disclosure provides a recombinant polypeptide comprising a resistin trimerization partner that advantageously and unexpectedly provides increased stability to the resulting trimeric recombinant fusion protein compared to fusions with commonly used trimerization domains T4Fib and GCN4, as shown in FIG. 8. In an embodiment the trimerization partner that confers stability to the recombinant fusion protein comprises the amino acid sequence set forth in SEQ ID NO: 1 or comprises an amino acid sequence having at least 85%, at least 90%, or at least 95% identity to the full length of the amino acid sequence set forth in SEQ ID NO: 1. In an embodiment, the trimerization partner comprises an amino sequence having at least 85% identity to the full length of the amino acid sequence set forth in SEQ ID NO: 2 and comprises a trimerization domain having at least 85% identity to SEQ ID NO: 1. In an embodiment, the trimerization partner comprises a trimerization domain having at least 85%, at least 90%, at least 95% or 100% identity to the full length of the amino acid sequence set forth in SEQ ID NO: 1 and the trimerization partner has at least 85%, at least 90%, at least 92%, at least 94%, at least 96%, at least 98%, or 100% identity to the full length of the amino acid sequence set forth in SEQ ID NO: 2. In an embodiment, the trimerization partner has at least 85%, at least 90%, at least 92%, at least 94%, at least 96%, at least 98%, or 100% identity to the full length of the amino acid sequence set forth in SEQ ID NO: 2. In an embodiment, the trimerization partner consists of the amino acid sequence set forth in SEQ ID NO: 2. In an embodiment, the trimerization partner comprises a resistin polypeptide or a fragment thereof that is sufficient to enable trimerization. In an embodiment, the resistin polypeptide or fragment thereof is a mammalian resistin or fragment thereof. In an embodiment, the resistin or fragment thereof is a human resistin or fragment thereof.

The present inventors have shown that the use of a resistin polypeptide as a trimerization partner in a recombinant polypeptide with a polypeptide, particularly a trimeric viral surface antigen, and more particularly a SARS-CoV-2 spike ectodomain polypeptide, unexpectedly and advantageously allows for robust expression of the recombinant polypeptide and for self-assembly of the recombinant polypeptide into a stable homotrimeric protein. A representative three-dimensional structure of a recombinant polypeptide comprising a SARS-CoV-2 spike ectodomain polypeptide linked to a resistin polypeptide is shown on the right in FIG. 1.

Human resistin is produced as a 108 amino acid polypeptide, including a 20 amino acid signal sequence that is cleaved to produce a mature polypeptide that is 88 amino acids in length. The penultimate N-terminal residue of the mature resistin polypeptide is a cysteine residue that allows hexamer formation. The resistin polypeptide used in the recombinant polypeptides described herein lacks the N-terminal leucine and cysteine residues of the mature resistin polypeptide, to prevent hexamer formation. In a preferred embodiment, the trimerization partner comprises a trimerization domain of a resistin polypeptide or the trimerization partner is a resistin polypeptide or the trimerization partner is a polypeptide substantially equivalent thereto. In a preferred embodiment, the resistin polypeptide, or the trimerization domain thereof, is a human resistin polypeptide or comprises a human resistin trimerization domain. In other embodiments, the resistin polypeptide or trimerization domain is a non-human resistin or trimerization domain, such as a mammalian resistin polypeptide or trimerization domain.

The use of a trimerization partner is particularly advantageous when the protein in need of expression is a trimeric protein, such as a viral surface antigen, or a fragment thereof. The methods and constructs of the present disclosure allow for production of a stable trimeric antigen that is structurally equivalent to a trimeric SARS-CoV-2 spike protein. The provided recombinant polypeptide unexpectedly allows the formation of a highly stable trimer (as shown in FIG. 8).

In an embodiment, the trimeric viral surface antigen is a coronavirus spike protein or a fragment thereof. In a preferred embodiment, the trimeric viral surface antigen is a SARS-CoV-2 spike protein or a fragment thereof. In an embodiment, the SARS-CoV-2 spike polypeptide comprises the amino acid sequence set forth in SEQ ID NO: 3 or SEQ ID NO: 4. In an embodiment, the SARS-CoV-2 spike polypeptide comprises an amino acid sequence having at least 70%, at least 75%, at least 80%, at least 85%, at least 90%, at least 95%, at least 96%, at least 97%, at least 98%, at least 99%, or 100% identity to the full length of the amino acid sequence set forth in SEQ ID NO: 3 or SEQ ID NO: 4. In an embodiment, the SARS-CoV-2 spike polypeptide comprises one or more amino acid substitutions relative to the full length of the amino acid sequence set forth in SEQ ID NO: 4. In an embodiment, the SARS-CoV-2 spike polypeptide comprises one, up to two, up to three, up to four, up to five, up to six, up to seven, up to eight, up to nine, up to ten, up to 15, up to 20, or more amino acid substitutions, insertions, and/or deletions relative to the full length of the amino acid sequence set forth in SEQ ID NO: 4. In another embodiment, the SARS-CoV-2 spike polypeptide may comprise one or more mutations from a known SARS-CoV-2 variant, such as B.1.1.7, B.1.351, or P.1 (for example, as described in Miao et al, 2021 or Guo et al, 2021).

The trimerization partner and the polypeptide to be expressed may optionally be joined by a linker. The selection of an appropriate linker will be readily apparent to one skilled in the art, as evidenced, for example, by Chen et al (2014). Linkers included in recombinant polypeptides are generally classified in three groups: flexible linkers, rigid linkers, and cleavable linkers, and they are typically 2-40 amino acids in length, though other lengths may also be suitable. Flexible linkers are generally composed of small non-polar (e.g. Gly) or polar (e.g. Ser) amino acids, though other amino acids such as Thr, Ala, Lys, and Glu may be included to maintain flexibility and/or improve solubility. Rigid linkers comprise amino acids chosen to favour the adoption of α-helical structures, such as Pro and Lys together with Glu and Ala. Cleavable linkers are chosen to allow cleavage by an enzyme of choice, allowing two portions of a recombinant protein to be separated either in vitro or in vivo. The polypeptides described in the examples provided herein comprise a flexible linker having the sequence GTGG. However, the linker could be omitted or an alternate linker sequence could be employed, as will be understood by one skilled in the art.

The recombinant polypeptide may optionally comprise one or more affinity tags to allow for ease of purification of the expressed protein. The selection of one or more affinity tags will be understood to one skilled in the art. Commonly used affinity tags include, for example, polyhistidine (commonly hexahistidine), FLAG, Streptag II, streptavidin-binding peptide (SBP), calmodulin-binding peptide (CBP), glutathione S-transferase (GST), maltose-binding protein (MBP), S-tag, HA-tag, and c-Myc tag. Depending on the nature of the affinity tag and the nature of the polypeptide being expressed, an affinity tag may be positioned at the N-terminus or C-terminus of the polypeptide. The polypeptides described in the examples provided herein include a FLAG tag and a 6×His tag at the C-terminus of each polypeptide. However, the inclusion of one or more affinity tags in the recombinant polypeptide is optional, and one or more other affinity tags could be employed, as will be understood to one skilled in the art.

A schematic of a recombinant polypeptide comprising a polypeptide of interest, a linker, a resistin polypeptide, and one or more affinity tags is shown in FIG. 2. Depending on the polypeptide of interest, the linker may be optional. Similarly, one or more affinity tags are included to allow ease of purification, but the affinity tags may be omitted if purification using affinity tags is not required.

A recombinant polypeptide as described herein may be produced using any suitable host cell and expression system. Commonly used host cells include prokaryotic expression host cells, such as E. coli, and eukaryotic host cells, such as yeast or cultured mammalian cells. The expression system may be a transient expression system or a stable expression system, and expression may be constitutive or inducible, depending on the promoter and/or other regulatory element(s) selected to drive expression. Suitable cells, vectors, promoters, and expression protocols will be well understood to one skilled in the art (for example, see Lalonde, 2017). For example, suitable mammalian cells include, but are not limited to, HEK293 cells, CHO cells, Vero cells, BHK cells, and CAP cells. In an embodiment, the host cell is a CHO cell. In an embodiment, the promoter is an inducible promoter. In an embodiment, the promoter is a cumate responsive promoter. Other suitable inducible promoters will be known to one skilled in the art and include, for example, a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter. A constitutive promoter may also be used, such as but not limited to, a cytomegalovirus (CMV) immediate early promoter, an elongation factor 1-alpha (EF1a) promoter, a cytomegalovirus enhancer/chicken β-actin (CAG) promoter, a simian vacuolating virus 40 (SV40) promoter, a phosphoglycerate kinase (PGK1) promote, or a ubiquitin C (Ubc) promoter.

A recombinant polypeptide or trimeric protein as described herein may be comprised in a composition together with a carrier. The carrier may be a pharmaceutically acceptable carrier, as will be understood to one skilled in the art. The composition may be an immunogenic composition and, in some embodiments, the composition may further comprise an adjuvant for enhancing an immune response in a subject. Suitable adjuvants will be understood to one skilled in the art and include, for example, aluminum salts, virosomes, water-in-oil emulsions such as MF59 and AS03, 6′-sulfate-β-D-Galp-(1,4)-β-D-Glcp-(1,1)-archaeol (SLA), monophosphoryl lipid A (MPL), QS-21, CpG oligodeoxynucleotide (CpG), polyinosinic-polycytidylic acid (Poly(I:C)), and liposomes, among others. In embodiments, an immunogenic composition may comprise a combination of two or more adjuvants.

A recombinant polypeptide or trimeric protein as described herein may be used as an antigen, for example in a vaccine, immunogenic composition, or serological assay.

A recombinant polypeptide or trimeric protein as described herein may be used in a method of identifying whether a subject has developed an antibody response to a pathogen or an antigen from a pathogen, the method comprising: contacting serum or a serum fraction from the subject with the recombinant polypeptide or trimeric protein and detecting binding of the recombinant polypeptide or trimeric protein by an antibody present in the serum or the serum fraction, wherein the second portion of the recombinant polypeptide comprises an amino acid sequence from an antigen of the pathogen. The pathogen may be SARS-CoV-2 and the recombinant polypeptide may comprise a SARS-CoV-2 spike ectodomain polypeptide as described herein. Methods to detect binding of a polypeptide or protein by an antibody would be known to one skilled in the art and include well established techniques such as western blotting and ELISA. The presence of an antibody against a pathogen or an antigen from a pathogen in the serum of a subject indicates that the subject has, at some time, been sufficiently exposed to the pathogen, or the antigen from the pathogen, to allow the subject to mount an antibody response; for example a subject may be exposed by infection and/or by vaccination.

A recombinant polypeptide or trimeric protein as described herein may be immobilized on a surface. For example, the recombinant polypeptide or trimeric protein may be covalently or non-covalently bonded to a surface. Methods for immobilizing proteins and surfaces suitable for protein immobilization are well known in the art. Examples of such surfaces include, but are not limited to: plastic; organosilane-derivatized glass; affinity chromatography media; protein-binding membranes, such as nitrocellulose, nylon, and polyvinylidene difluoride (PVDF) membranes; and hydrogels.

A surface-immobilized recombinant polypeptide or trimeric protein as described herein, may be comprised by a device. Suitable devices will be known to one skilled in the art include, but are not limited to: plates, such as microwell or microtiter plates; affinity chromatography columns; microarrays or biochips; microfluidic devices; and test cassettes, such as blood or serum test cassettes. A recombinant polypeptide or trimeric protein as described herein may be included in a kit, together with an antigen-binding molecule that specifically binds the recombinant polypeptide or trimeric protein. The antigen-binding molecule may be, but is not necessarily, an antibody or an antigen-binding fragment thereof. In some embodiments, the antigen-binding molecule may bonded to a detectable label to allow detection of the presence of the antigen-binding molecule and, correspondingly, allow detection of the presence of the recombinant polypeptide or trimeric protein when the recombinant polypeptide or trimeric protein bound by the antigen-binding molecule.

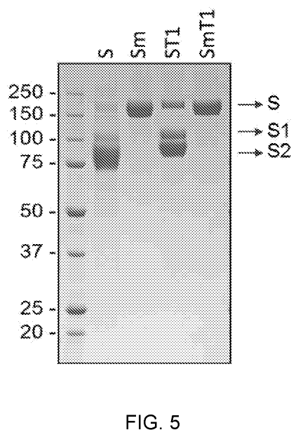

The recombinant polypeptide described herein advantageously allows for the production and purification of the corresponding homotrimer (as shown in FIG. 5).

The provided recombinant polypeptide may advantageously leverage the anti-apoptotic, anti- and pro-inflammatory, and non-immunogenic nature of resistin.

The present disclosure provides a trimerization partner that unexpectedly and advantageously allows for stable trimerization of the polypeptide in solution.

### EXAMPLES

The following non-limiting examples are illustrative of the present disclosure.

### Example 1: Preparation of SARS-CoV-2 Spike Protein Expression Constructs

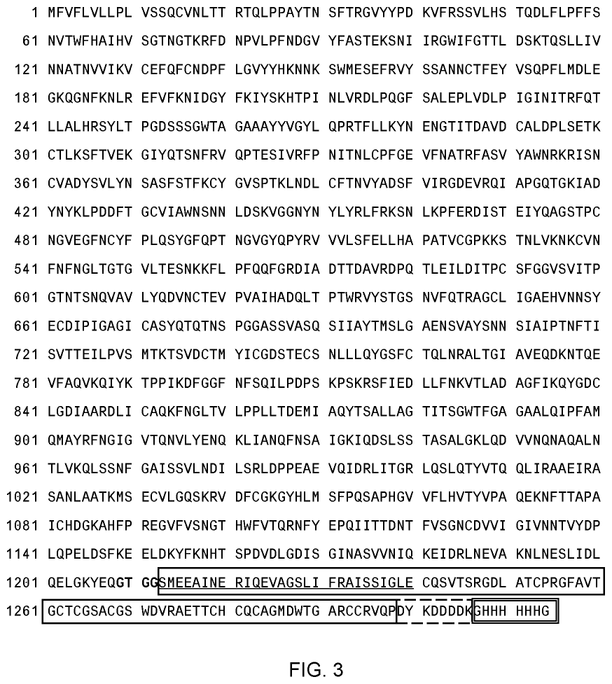

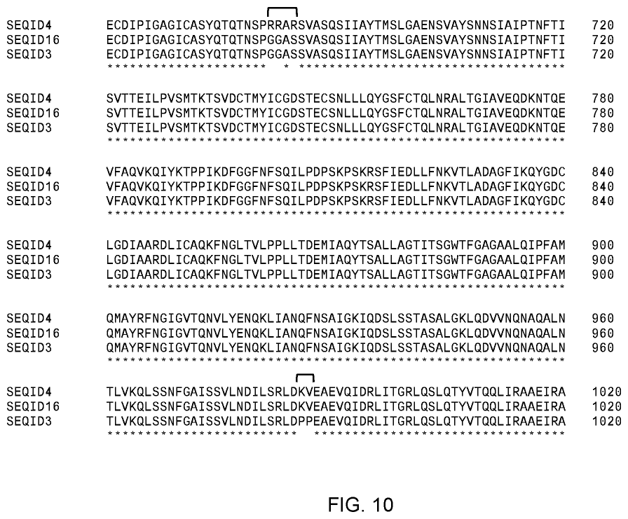

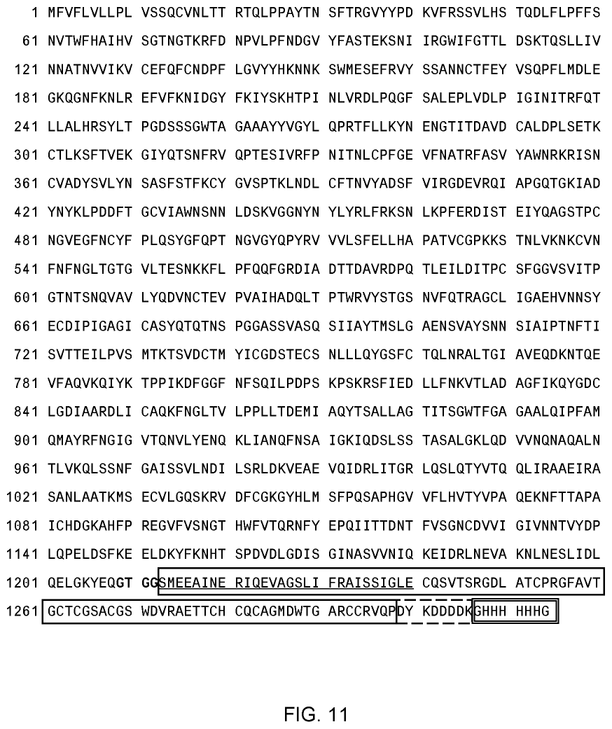

A codon-optimized (Cricetulus griseus codon bias) SARS-CoV-2 spike (S) protein cDNA (aa 1-1208) was synthesized by GenScript and cloned into the pTT®5 plasmid expression vector, under control of a CMV promoter. Three versions of the S protein were expressed: one encoding an unmodified ectodomain (ECD) protein sequence (S), having the amino acid sequence set forth in SEQ ID NO: 4; a second encoding a modified ECD sequence (Sm) with two modifications, one to prevent furin-mediated S1/S2 cleavage (RRAR->GGAS) and the second to stabilize the pre-fusion conformation (K986P V987P), as recently described (Wrapp et al. 2020), having the amino acid sequence set forth in SEQ ID NO: 3; and a third encoding a second modified ECD sequence (Sm2) with one modification to prevent furin-mediated S1/S2 cleavage (RRAR->GGAS), having the amino acid sequence set forth in SEQ ID NO: 16. The locations of the RRAR->GGAS mutation in Sm and Sm2 and the K986P and V987P mutations in Sm are shown in the sequence alignment provided in FIG. 10. To mimic the native trimeric structure of the spike protein, as found on SARS-CoV-2 virus particles, constructs were prepared with three different trimerization partners fused to the C-terminus of the ectodomain in various constructs (ST1, ST2, ST3; SmT1, SmT2 and SmT3; and Sm2T1). The sequence of SmT1 is shown in FIG. 3. The sequence of Sm2T1 is shown in FIG. 11. Sequences for all constructs are provided in the sequence listing.

The first trimerization partner tested was a resistin polypeptide having the amino acid sequence set forth in SEQ ID NO: 2 (“T1”), the second trimerization partner tested was a T4 phage fibritin or “foldon” trimerization domain (“T2”) having the amino acid sequence set forth in SEQ ID NO: 9, and the third trimerization partner tested was a yeast GCN4 trimerization domain (“T3”) having the amino acid sequence set forth in SEQ ID NO: 10. Each construct includes FLAG and 6×His tags for purification, cloned in-frame at, or proximal to, the C-terminus of the S, Sm, or Sm2 polypeptide.

### Example 2: Expression of SARS-Cov-2 Spike Protein

The present inventors assessed the potential of three polyethylenimine (PEI)-mediated transient expression platforms for production of the SARS-CoV-2 spike constructs: two methods, based on EBNA1-expressing CHO cells (CHO-3E7) (Stuible et al. 2018) and HEK293 cells (293-6E) (L′Abbé et al. 2018) have served as core platforms for recombinant protein production by the present inventors for several years, and generally perform very well for a wide range of recombinant antibodies and other proteins. For both methods, cells were cultured in chemically-defined F17 media and transfected at low cell density using PEI. A third method used a CHO-DXB11-derived clone (CHO^{BRI/55E1}) that expresses machinery for cumate-inducible protein expression (Poulain et al. 2017; Poulain et al. 2019); CHO^{BRI/55E1} cells are routinely used by the present inventors for stable cell line development for biologics manufacturing.

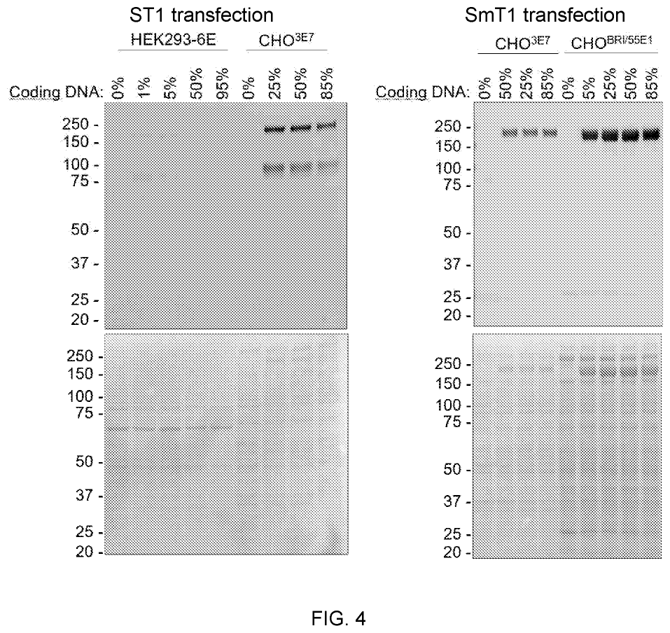

Preliminary expression analysis at day 5 post-transfection indicated that the CHO cells allowed secretion of 5- to 20-fold higher levels of S protein compared to HEK293 cells (FIG. 4), so further process optimization was done using CHO cells. Similar expression tests were performed for the ST1, SmT1, and Sm2T1 constructs using the CHO-3E7 and CHO^{BRI/55E1} platforms. As shown in FIG. 4, the CHO^{BRI/55E1} platform gave substantially higher yields, estimated at 70-120 mg/L based on a purified and quantified SmT1 standard (not shown). This yield is 35-100-fold higher than the typical titers of 0.5-2 mg/L reported in the literature for expression of coronavirus spike protein using transient HEK293 transfection.

### Example 3: Purification of SARS-Cov-2 Spike Protein

The present inventors developed a purification process using immobilized metal affinity chromatography (IMAC) followed by an anti-FLAG affinity column. A third polishing step using preparative size-exclusion chromatography was used to obtain highly pure protein preparations (FIG. 5). Purified proteins are estimated to be >98% pure on Coomassie®-stained SDS-PAGE gels (FIG. 5). The ST1 protein shows 3 bands on the gel, indicating significant level of cleavage at the furin site during expression, generating the S1 and S2 subdomains, with some uncleaved S remaining. In contrast, the SmT1 protein shows a single ˜180 kDa band (FIG. 5) as expected for a highly glycosylated protein.

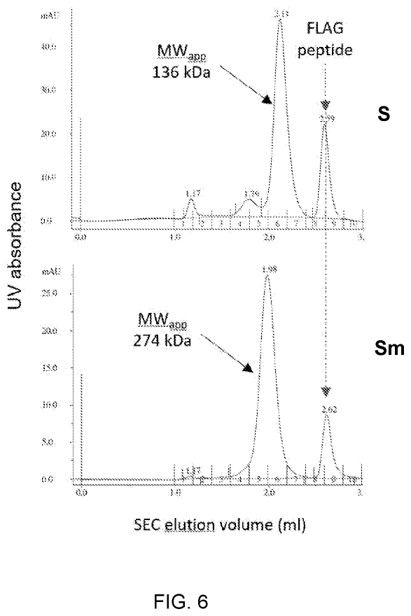

The purified recombinant S and Sm proteins were analyzed by analytical size exclusion chromatography (SEC) on Superose® 6 5/15 column (FIG. 6). The elution profiles indicate that neither the S nor the Sm proteins are able to form stable trimers in solution based on their apparent molecular weight (136 and 274 kDa, respectively). The lower apparent molecular weight of S may be explained by the loss of the cleaved S1 fragment during affinity purification, leaving only the S2 subdomain as the predominant species in the 136 kDa peak.

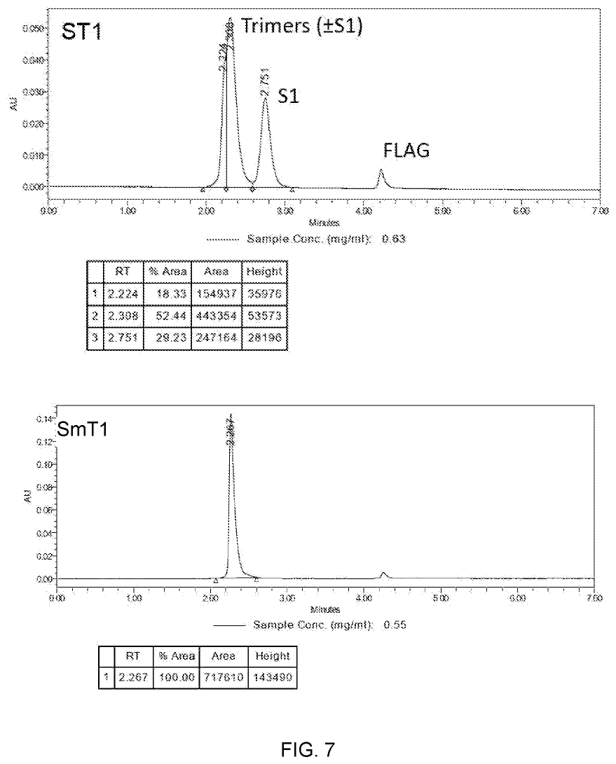

Analysis of the affinity purified resistin fusion constructs (ST1 and SmT1) was done by SEC-UPLC coupled to a MALS detector (FIG. 7). The elution profile obtained with the ST1 construct (upper panel) clearly indicates the presence of three species, one minor (18%) corresponding to the unprocessed S protein, one major (52%) corresponding to the S2 trimer lacking the S1 fragment, and a third peak (29%) corresponding to the S1 fragment. In contrast, the SmT1 construct (lower panel) elutes as a single symmetric peak that correspond to a 100% pure trimer with a calculated mass of 486 kDa.

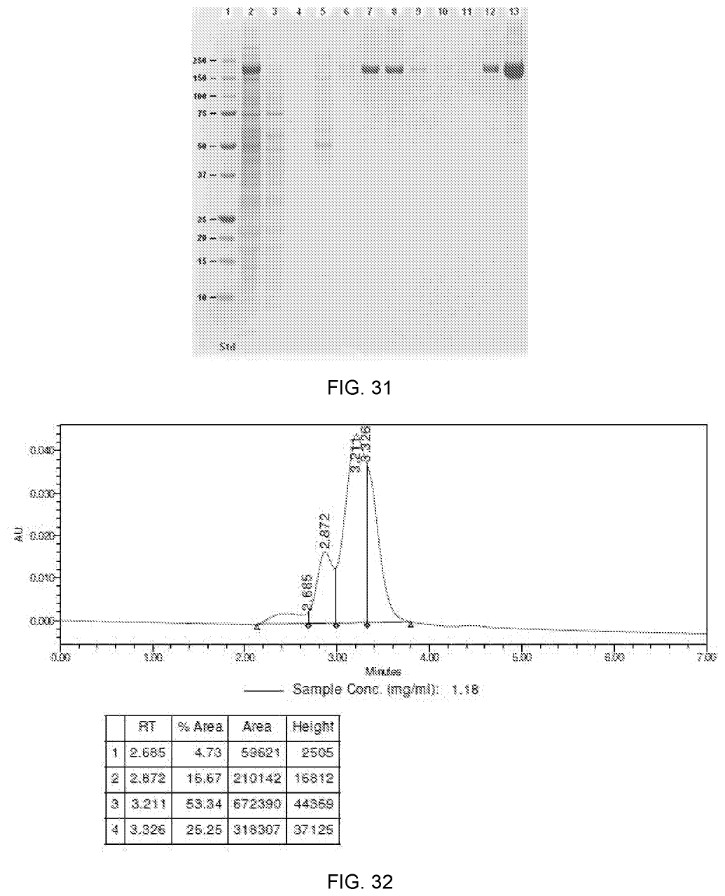

The Sm2T1 protein was expressed from a stable cumate-inducible CHO pool. Culture medium was harvested a day 7 post-induction and clarified by centrifugation and filtration. Clarified medium was purified by IMAC, as described above for SmT1, and formulated in DPBS. A typical purification process result is exemplified by the SDS-PAGE gel shown in in FIG. 31.

Analysis of the affinity purified Sm2T1 resistin fusion construct was done by SEC-UPLC coupled to a MALS detector (FIG. 32). The elution profile obtained with the Sm2T1 construct indicates that Sm2T1 elutes as a mixture of high molecular weight species (21%) and trimeric species (79%).

### Example 4: Comparison of Resistin Fusion Partner to T4 Phage Foldon and Yeast GCN4 Trimerization Domains

For the sake of comparison, the SmT1 construct was compared to SmT2 (containing the T4 phage foldon trimerization domain) and SmT3 (containing the yeast GCN4 trimerization domain). The SEC-UPLC elution profiles (FIG. 8) indicate that SmT2 and SmT3 are heterogeneous trimers with the presence of some higher molecular weight aggregates (SmT2) and some tailing species (SmT2 and SmT3) that may indicate the presence of partially unfolded protein. In contrast, the elution profile of the SmT1 construct indicates that it is a substantially homogeneous trimer and there is no evidence of trailing species, suggesting that substantially all of the SmT1 the protein is properly folded.

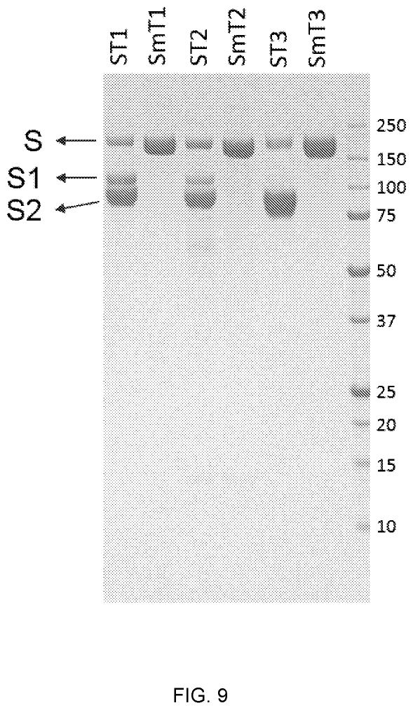

Affinity purified S proteins were analyzed by reducing SDS-PAGE and stained with Coomassie® blue (FIG. 9). Molecular weight protein standards are shown in the right-most lane. From the gel data, it is clear that the ST2 construct lost significant amounts of the cleaved S1 fragment during purification, while for ST3 the loss was almost complete. Similar to ST2, ST1 also shows significant loss of the cleaved S1 fragment. For SmT1, SmT2 and SmT3, no clear qualitative differences between the constructs could be observed on the gel and none of these constructs shows evidence of furin-mediated S1/S2 cleavage.

### Example 5: Immunogenicity of SARS-CoV-2 Spike Protein-Based Vaccine Formulations

To evaluate the immunogenicity of SmT1 in a vaccine setting, male (n=8) and female (n=8 for all groups except group 5, in which n=7) C57Bl/6 mice were immunized with 2 or 10 μg of the protein alone or in combination with various adjuvants on days 0 and 21 (see Table 1 for a list of adjuvants used). Serum was collected on Day 20 and Day 28 for evaluation of antigen-specific IgG responses by ELISA. In addition, splenocytes were collected on Day 28 and assayed by ELISpot and intracellular cytokine staining (ICCS) for the evaluation of antigen-specific T cell responses. Cells were stimulated with peptide pools (15-mers overlapping by 11 a.a.) that cover the entire length of the spike protein (split into two N- or C-terminal pools) or with SmT1 protein. The secretion of cytokines such as IFN-γ, IL-2 and TNF-α in response to antigen stimulation was indicative of an antigen specific T cell response.

TABLE 1
Vaccine formulations of SmT1 used to immunize mice

| Group (n = 15-16) | Adjuvant | SmT1 Dose |
|---|---|---|
| 1 | None | 10 μg |
| 2 | SLA | 2 μg |
| 3 | Adju-Phos ® | |
| 4 | MPL/QS-21/Liposomes | |
| 5 | SLA | 10 μg |
| 6 | Adju-Phos ® | |
| 7 | MPL/QS-21/Liposomes | |

SLA = 6′-sulfate-β-D-Galp-(1,4)-β-D-Glcp-(1,1)-archaeol;
MPL = monophosphoryl lipid A;
AdjuPhos ® = aluminum phosphate

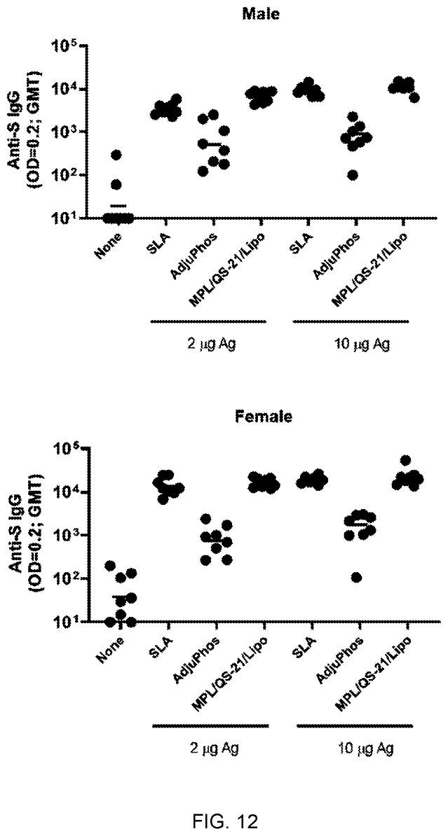

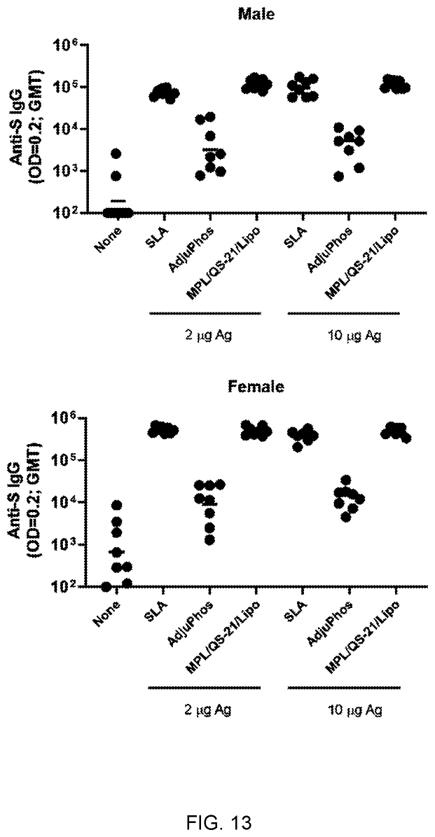

When combined with the various adjuvants, as detailed in Table 1, SmT1 was capable of inducing a strong IgG response in both male and female mice after a single immunization (FIG. 12). When adjuvanted with SLA or Liposomes/QS-21/MPL formulations, antibody levels were >100-fold higher than antigen alone, even when 5-fold less protein antigen was administered. Antibody levels were generally similar in animals receiving 2 or 10 μg of adjuvanted SmT1. Antibody titers were further increased following a second immunization with a >10-fold increase in antibody titers seen with all vaccine formulations (FIG. 13). The IgG1/IgG2c bias in the antibody response was also assessed, with most formulations showing a bias towards IgG1, while the inclusion of Liposomes/QS-21/MPL shifted the response more towards IgG2c (FIG. 14).

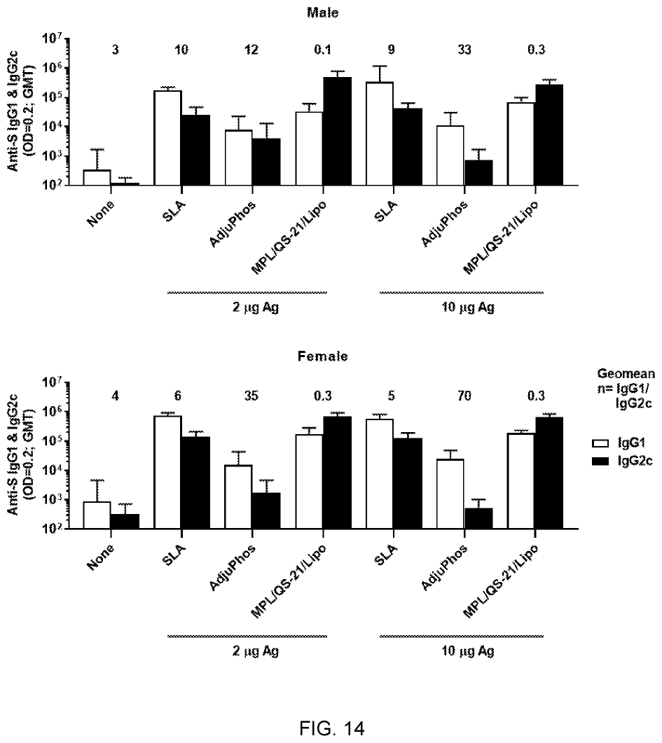

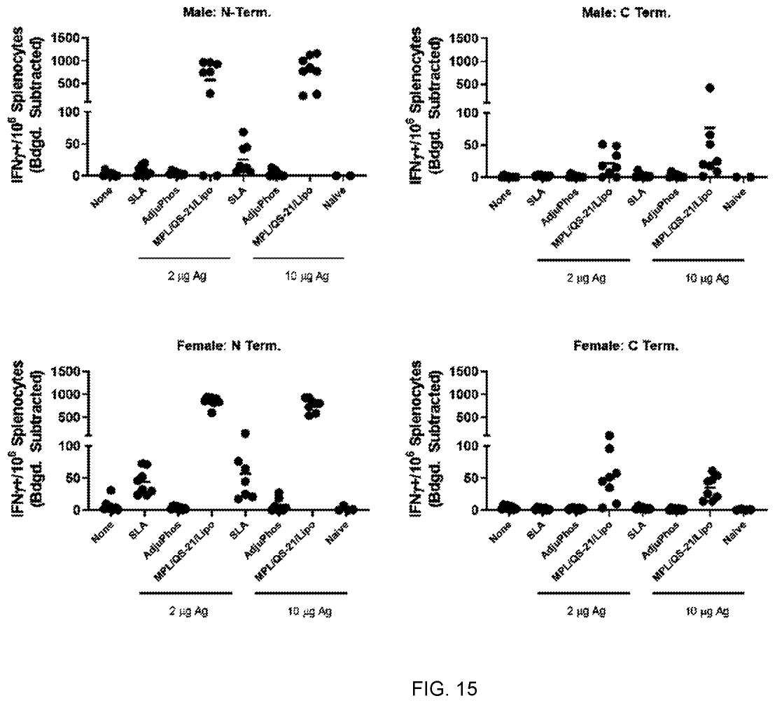

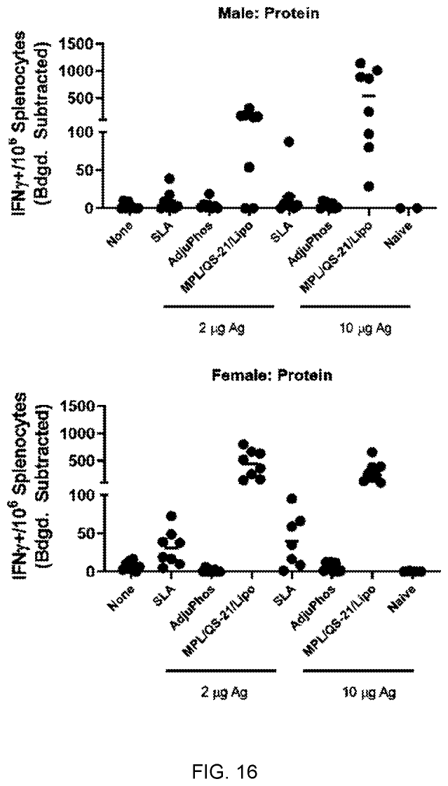

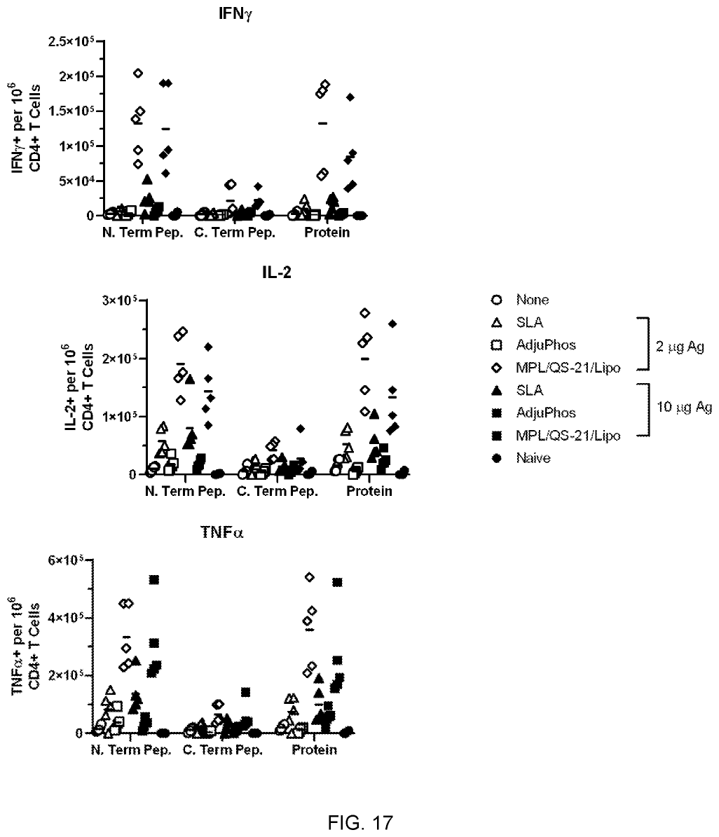

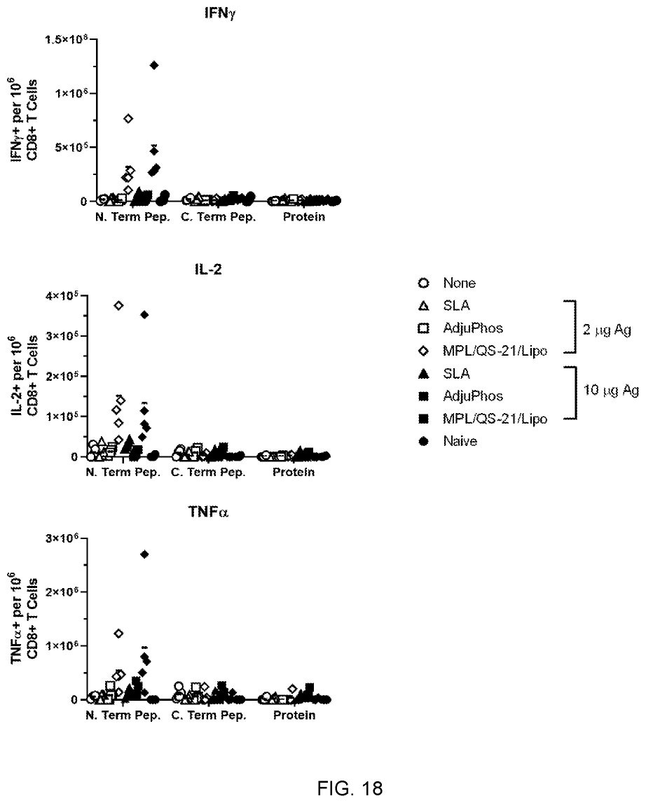

SmT1 was also capable of inducing antigen-specific T cell responses when combined with certain adjuvants as demonstrated by IFN-γ ELISpot (FIGS. 15 & 16). The inclusion of SLA or in particular Liposomes/QS-21/MPL in the vaccine formulations led to an increase in the number of IFN-γ+ spot-forming cells upon stimulation with the Spike peptide pools or whole SmT1 protein. These responses were detected in mice administered either the 2 or 10 μg antigen dose. In addition, ICCS analysis in a subset of the female mice revealed that these T cells consisted of both CD4 and CD8 T cells (FIGS. 17 & 18, respectively). Both types of cells contained increased levels of IL-2 and TNF-α in addition to IFN-γ upon stimulation with spike protein or peptide pools.

Vaccine compositions comprising lower doses (1 μg and 0.1 μg) of SmT1 antigen in combination with various adjuvants were also tested (see Table 2 for a list of vaccine formulations). In addition, vaccine compositions comprising Sm2T1 in combination with various adjuvants (see Table 2 for a list of vaccine formulations) were tested to compare the immunogenicity of Sm2T1 to that of SmT1.

TABLE 2
Vaccine formulations of SmT1 and SmT2 used to immunize mice

| Group | Adjuvant | Antigen |
|---|---|---|
| 1 | None | SmT1 (1 μg) |
| 2 | SLA | |
| 3 | CpG | |
| 4 | Poly(I:C) | |
| 5 | SLA + CpG | |
| 6 | SLA + Poly(I:C) | |
| 7 | SLA | SmT1 (0.1 μg) |
| 8 | CpG | |
| 9 | Poly(I:C) | |
| 10 | SLA + CpG | |
| 11 | SLA + Poly(I:C) | |
| 12 | None | Sm2T1 (1 μg) |
| 13 | SLA | |
| 14 | AdjuPhos ® | |
| 15 | AS01_{B} | |
| 16 | Poly(I:C) | |

SLA = 6′-sulfate-β-D-Galp-(1,4)-β-D-Glcp-(1,1)-archaeol; CpG = CpG oligodeoxynucleotide, Poly(I:C) = polyinosinic-polycytidylic acid, SLA = sulfated lactosylarchaeol, AdjuPhos ® = aluminum phosphate, AS01_{B} = AS01B adjuvant system.

Female C57Bl/6 mice (n=10/group) were immunized on days 0 and 21 (with vaccine formulations as described in Table 2). Serum was collected on Day 20 and Day 28 for evaluation of antigen-specific IgG responses by ELISA and neutralization assay, respectively. In addition, splenocytes were collected on Day 28 and assayed by ELISpot for the evaluation of antigen-specific T cell responses. Cells were stimulated with peptide pools (15-mers overlapping by 11 a.a.) that cover the entire length of the spike protein (split into two N- or C-terminal pools) or with SmT1 protein.

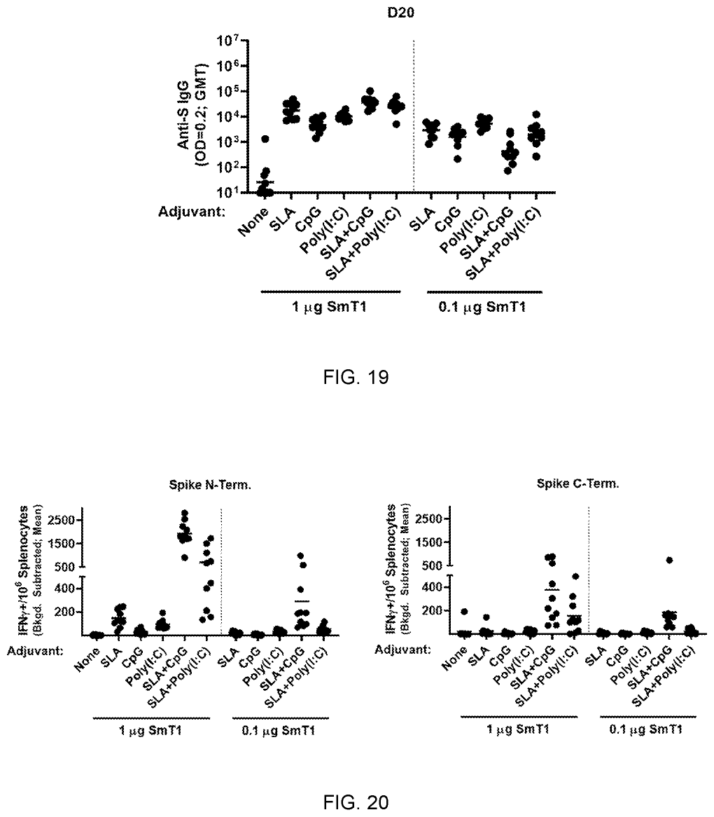

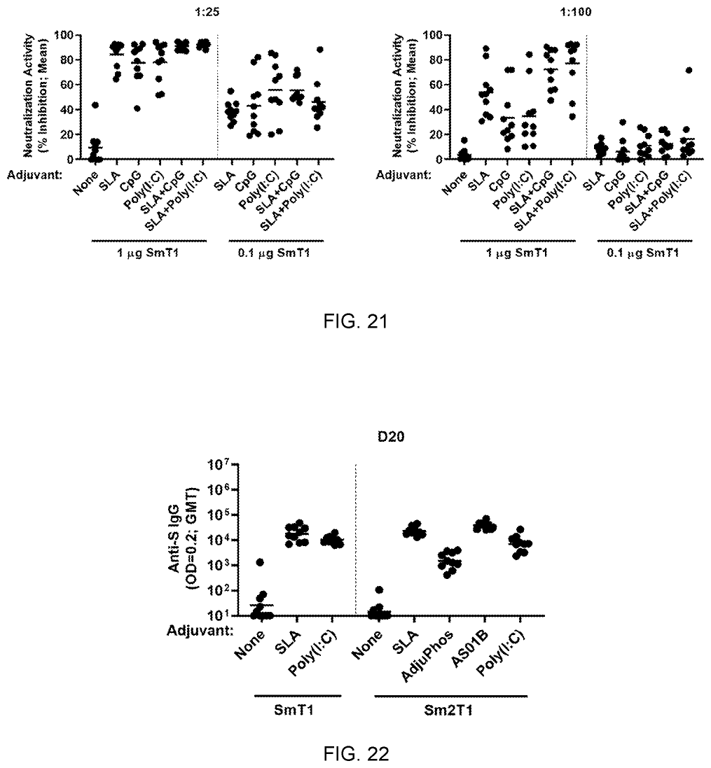

When combined with the various adjuvant formulations, SmT1 at either 0.1 or 1 μg dose was capable of inducing a strong IgG response after a single immunization (FIG. 19). While antigen-specific T cell responses were detected by IFN-γ ELISpot in the splenocytes of mice immunized with SmT1 adjuvanted with SLA or Poly (I:C), an >5-fold increase in IFN-γ+splenocytes was observed in mice immunized with SmT1 adjuvanted with the SLA+CpG or SLA+Poly(I:C) combination adjuvant formulations (FIG. 20). The functionality of the antigen-specific antibodies was measured in a surrogate neutralization assay, where the ability of immunized mouse serum collected on Day 28 to inhibit the binding of labeled soluble spike protein to VERO cells was assessed. A clear antigen dose effect was evident. While serum (diluted 1 in 75) from mice immunized with antigen alone induced low neutralization activity, adjuvanted formulations induced ˜40% and ˜80% neutralization with 0.1 and 1 μg of SmT1, respectively (FIG. 21). When a more stringent 1:300 serum dilution was used, neutralization was still clearly detected in the serum of mice immunized with adjuvanted formulations of 1 μg SmT1. As with the T cell responses, the SLA+CpG and SLA+Poly(I:C) combination formulations showed superior activity (>70% neutralization) in this assay.

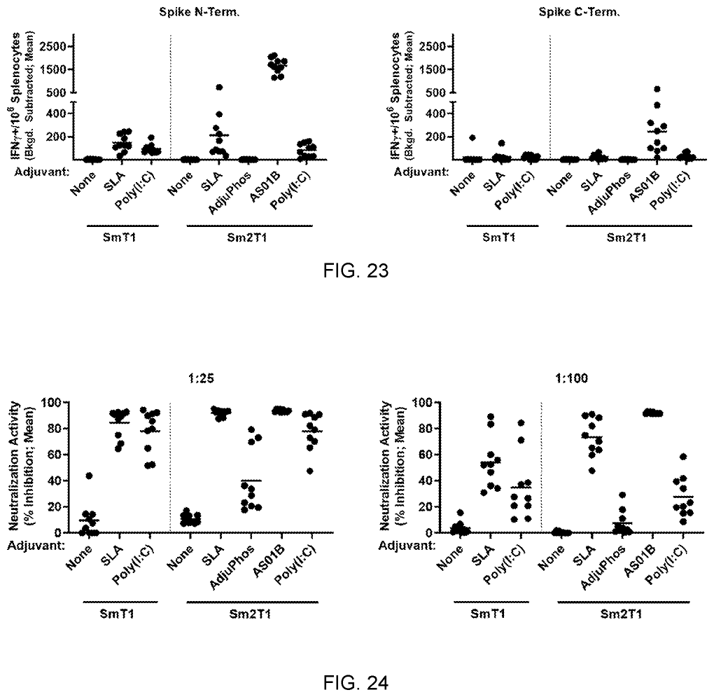

The same set of assays were utilized to evaluate the immunogenicity of Sm2T1 when combined with different types of adjuvants. As with SmT1, Sm2T1 when combined with an appropriate adjuvant was capable of inducing strong antigen-specific antibody (FIG. 22) and cellular responses (FIG. 23). In addition, the immunized mouse serum was able to inhibit binding of SARS-CoV-2 Spike protein in the surrogate neutralization assay (FIG. 24). The level of the measured response was dependent on the type of adjuvant used. Interestingly, similarly adjuvanted formulations of SmT1 and Sm2T1 showed equal levels of immunogenicity (both humoral and cellular) when compared head-to-head.

Vaccine compositions comprising 3 μg of SmT1 antigen in combination with various adjuvants were tested in a SARS-CoV-2 hamster challenge model (see Table 3 for a list of groups).

TABLE 3
Vaccine formulations of SmT1 used to immunize hamsters

| | | | Vaccination |
|---|---|---|---|
| Group | Adjuvant | Antigen | Timepoints |
| 1 | None | None | Days 0 and 21 |
| 2 | SLA | | |
| 3 | SLA + CpG | | |
| 4 | None | SmT1 (3 μg) | |
| 5 | SLA | | |
| 6 | CpG | | |
| 7 | SLA + CpG | | |
| 8 | SLA | SmT1 (3 μg) | Day 0 |
| 9 | SLA + CpG | | |

SLA = sulfated lactosylarchaeol = 6′-sulfate-β-D-Galp-(1,4)-β-D-Glcp-(1,1)-archaeol; CpG = CpG oligodeoxynucleotide.

Female Syrian Golden hamsters (n=6/group) were immunized on days 0 and/or 21 with SmT1 alone or with adjuvant (as described in Table 3). As negative controls, animals were immunized with vehicle (group 1) or adjuvant alone (groups 2 and 3). Serum was collected on Day 34 for evaluation of antigen-specific IgG responses by ELISA and neutralization assay. On Day 35, animals were challenged intranasally with 10⁵ plaque forming units of live SARS-CoV-2 virus. The effect of viral challenge of body weight was tracked for 5 days post viral challenge. On Day 40, animals were euthanized and lungs collected for the quantification of viral load by plaque assay.

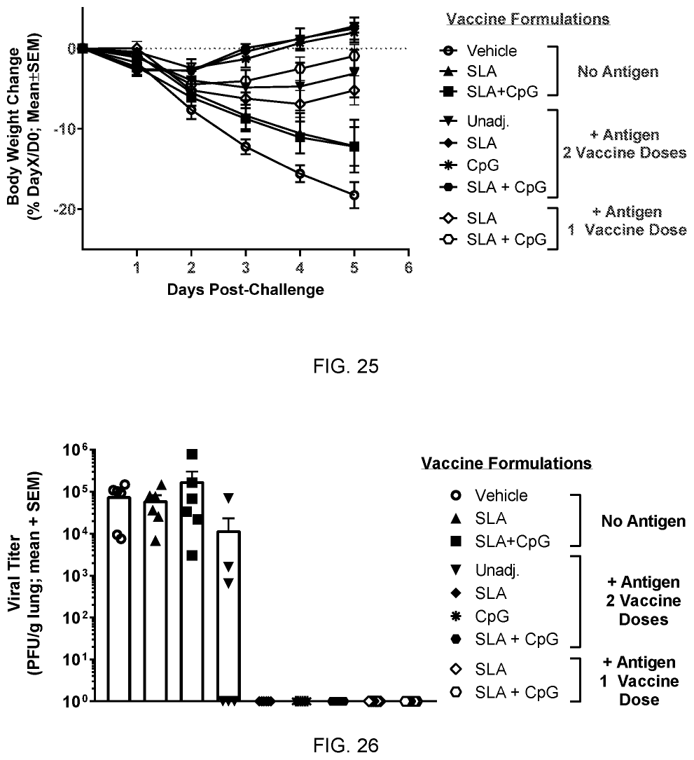

Animals immunized with vehicle or adjuvant alone continued losing weight during the course of the study (FIG. 25). SmT1 was able to protect the hamsters from viral challenge, as prior treatment with any of the vaccine regimens containing SmT1 antigen led to a significant decrease in body weight loss. The adjuvanted prime/boost regimens greatly protected the hamsters with their average body weight loss never exceeding 3% and a steady gain in body weight seen thereafter. While not as effective, a decrease in body weight loss was also observed with the single dose vaccine regimens or the double dose of antigen alone. The ability of the vaccine formulations to protect from viral infection was confirmed when measuring the viral load in the lungs on Day 5. Average viral loads >60,000 PFU/g lung tissue were seen in hamsters treated with vehicle or adjuvant alone (FIG. 26). No detectable viral titers were observed in lungs of any of the hamsters immunized with adjuvanted Spike mECD formulations (single or double vaccinations). The antigen alone formulation did lead to a significant decrease in average viral titers, but virus was still detectable in 3/6 animals.

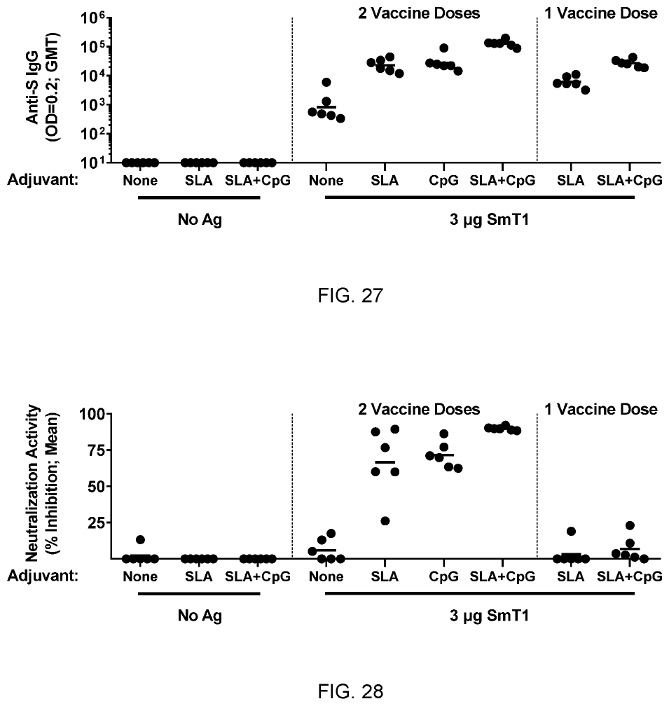

Anti-Spike IgG titers were observed in the serum of all hamsters immunized with antigen alone following a single or double vaccine dose (FIG. 27). Titers were ˜1-log higher in animals that received the 2^{nd} vaccination on Day 21 as compared to those that received a single dose of similarly-adjuvanted SmT1 formulation on Day 0. The activity of the immunized hamster serum was confirmed in the cell-based neutralization assay (FIG. 28). When serum was diluted to 1:75, the serum from animals receiving two doses of Spike mECD with SLA, CpG or SLA+CpG had significantly higher neutralization (>66%) than other groups.

These results demonstrate that an antigen comprising a coronavirus spike ectodomain fused to a resistin polypeptide is immunogenic when combined with various adjuvants and that this antigen is able to generate strong humoral and cell-mediated immune responses.

### Example 6: INSTI® Serological Assay Using SmT1 or Sm2T1 Antigen

INSTI® Test Device production: INSTI® blotted membrane units (BMUs) were blotted with SmT1 at a concentration of 1.21 mg/mL or Sm2T1 at a concentration of 1.19 mg/mL.

INSTI® Test Procedure: 50 μL of serum/plasma was added to a bottle of INSTI® Solution 1, mixed by inversion, and poured onto the INSTI® test device. Once the liquid has flowed through the device (<20 sec), INSTI® Solution 2 (Color Developer) is poured into the device, followed by INSTI® Solution 3 (Clarifying Solution). Total test time was ˜60 seconds. A positive result is obtained if a blue dot appears in the area where a specific antigen was blotted.

Sensitivity challenge: To test analytical sensitivity, one limit of detection sample (LoD) was tested at n=20 replicates. To test clinical sensitivity, 10 unique COVID-19 positive samples were tested at n=1.

Specificity challenge: 33 samples were tested at n=1. Samples were positive for other medical conditions such as Hepatitis B (2), pregnancy (8), Epstein-Barr Virus (2), HIV (5), Syphilis (2), Hepatitis C (2), Influenza vaccination (2), and human Coronavirus (10).

Results:

TABLE 4
Comparison of COVID-19 SmT1 and COVID-19 Sm2T1 results

| | Reactive Results - | Reactive Results - | Reactive Results - |
|---|---|---|---|
| Antigen | LoD Sample | Positive Samples | Negative Samples |
| SmT1 | 20/20 | 9/10 | 0/33 |
| Sm2T1 | 20/20 | 9/10 | 0/33 |

When testing the LoD sample, both SmT1 and Sm2T1 were able to detect all 20 replicates (see Table 4). Therefore, these antigens appear to be equivalent in terms of analytical sensitivity.

In the clinical sensitivity and specificity evaluations, both antigens detected the same nine COVID-19 positive specimens and were unable to detect antibodies for the remaining one sample. Therefore the two antigens appear to be equivalent in terms of clinical sensitivity. For the 33 COVID-19 negative samples, including samples positive for human coronavirus, no false positives were observed for either antigen (see Table 4).

### Example 7: Testing Resistin as a Trimerization Partner for VHH

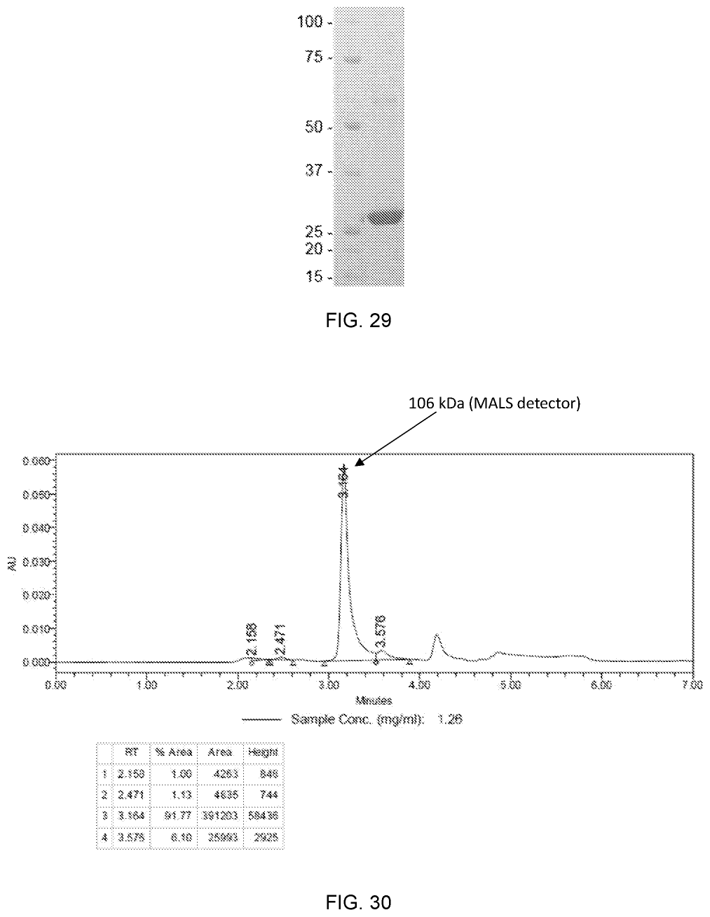

To test whether resistin is an effective trimerization partner for other proteins, a VHH antibody (aka nanobody) was fused in-frame with the human resistin gene and the resulting construct was cloned into the pTT® 5 plasmid, followed by transfection into CHO cells. Following transient expression in CHO cells, the VHH-RSTN fusion was purified by IMAC. Purified VHH-RSTN was analyzed by SDS-PAGE under reducing conditions (FIG. 29) and by SEC-UPLC (FIG. 30) linked to a MALS detector. The calculated molecular weight of the VHH-RSTN was 106 kDa, suggesting that it exists as a trimer in solution (based on the amino acid sequence, the expected molecular weight is 84 kDa).

The preceding examples have been provided to illustrate various aspects of the invention and are non-limiting. The scope of the claims is not limited to specific details provided in the examples; rather the claims are to be given the broadest interpretation consistent with the teachings of the specification and drawings as a whole.

### REFERENCES

The content of each of the following references is hereby incorporated by reference in its entirety.
Altschul, S. F., Gish, W., Miller, W., Myers, E. W., Lipman, D. J., Basic local alignment search tool, Journal of Molecular Biology, 1990, 215(3): 403-410.
Altschul, S. F., Madden, T. L., Schäffer, A. A., Zhang, J., Zhang, Z., Miller, W., Lipman, D. J., Gapped BLAST and PSI-BLAST: a new generation of protein database search programs, Nucleic Acids Research, 1997, 25(17): 3389-402.
Chen, X., Zaro, J. Shen, W.-C., Fusion Protein Linkers: Property, Design and Functionality, Advanced Drug Delivery Reviews, 65(10): 1357-1369.
Chun, J., et al., Effect of fc fusion on folding and immunogenicity of middle east respiratory syndrome coronavirus spike protein. Journal of microbiology and biotechnology, 2019, 29(5): p. 813-819.
Coleman, C. M., et al., Purified coronavirus spike protein nanoparticles induce coronavirus neutralizing antibodies in mice. Vaccine, 2014, 32(26): p. 3169-3174.
Du, L., et al., A 219-mer CHO-expressing receptor-binding domain of SARS-CoV S protein induces potent immune responses and protective immunity. Viral immunology, 2010, 23(2): p. 211-219.
Eisenberg, D., Schwarz, E. Komaromy, M., Wall, R. Analysis of membrane and surface protein sequences with the hydrophobic moment plot. Journal of Molecular Biology, 1984, 179: 125-142.
Gui, M., et al., Cryo-electron microscopy structures of the SARS-CoV spike glycoprotein reveal a prerequisite conformational state for receptor binding. Cell Research, 2017, 27(1): p. 119-129.
Guo, X., et al., The genetic variant of SARS-CoV-2: would it matter for controlling the devastating pandemic? International Journal of Biological Sciences, 2021, 17(6): 1476-1485.
Harbury P B, Zhang T, Kim P S, Alber T, A switch between two-, three-, and four-stranded coiled coils in GCN4 leucine zipper mutants. Science, 1993, 262, 1401-1407.
Huang, X., et al., Human coronavirus HKU1 spike protein uses 0-acetylated sialic acid as an attachment receptor determinant and employs hemagglutinin-esterase protein as a receptor-destroying enzyme. Journal of virology, 2015, 89(14): p. 7202-7213.
Kam, Y. W., et al., Antibodies against trimeric S glycoprotein protect hamsters against SARS-CoV challenge despite their capacity to mediate FcγRII-dependent entry into B cells in vitro. Vaccine, 2007, 25(4): p. 729-740.
Karlin, S., Altschul, S. F., Applications and statistics for multiple high-scoring segments in molecular sequences. PNAS, 1993, 90(12): 5873-5877.
Karlin, S., Altschul, S. F., Methods for assessing the statistical significance of molecular sequence features by using general scoring schemes. PNAS, 1990, 87(6): 2264-2268.
Kirchdoerfer, R. N., et al., Stabilized coronavirus spikes are resistant to conformational changes induced by receptor recognition or proteolysis. Scientific reports, 2018. 8(1): p. 1-11.
L′Abbé, D., et al., Transient gene expression in suspension HEK293-EBNA1 cells, in Recombinant Protein Expression in Mammalian Cells. 2018, Springer. p. 1-16.
Lalonde M E, Durocher Y, Therapeutic glycoprotein production in mammalian cells, Journal of Biotechnology, 2017, 251: 128-140.
Li, J., et al., Immunogenicity and protection efficacy of monomeric and trimeric recombinant SARS coronavirus spike protein subunit vaccine candidates. Viral immunology. 2013, 26(2): p. 126-132.
Miao, M. et al, Genetic diversity of SARS-CoV-2 over a one-year period of the COVID-19 pantemic: a global perspective. 2021, 9: 412.
Myers, E. W., Miller, W., Optimal alignments in linear space, Bioinformatics, 4(1): 11-17.Sliepen K, van Montfort T, Melchers M, Isik G, Sanders R W, 2015. Immunosilencing a highly immunogenic protein trimerization domain. J Biol Chem 290, 7436-7442.
Stuible, M., et al., Optimization of a high-cell-density polyethylenimine transfection method for rapid protein production in CHO-EBNA1 cells. Journal of biotechnology, 2018. 281: p. 39-47.
Tanokura M. 1H-NMR study on the tautomerism of the imidazole ring of histidine residues. I. Microscopic pK values and molar ratios of tautomers in histidine-containing peptides. Biochim Biophys Acta. 1983; 742:576-85.
Tao Y, Strelkov S V, Mesyanzhinov V V, Rossmann M G, 1997. Structure of bacteriophage T4 fibritin: a segmented coiled coil and the role of the C-terminal domain. Structure 5, 789-798.
Tortorici, M. A., et al., Structural basis for human coronavirus attachment to sialic acid receptors. Nature structural & molecular biology, 2019. 26(6): p. 481-489.
Walls, A. C., et al., Structure, function, and antigenicity of the SARS-CoV-2 spike glycoprotein. Cell, 2020, 181(2): 281-292.e6.
Wrapp, D., et al., Cryo-EM structure of the 2019-nCoV spike in the prefusion conformation. Science, 2020. 367(6483): p. 1260-1263.
Xia, S., et al., Inhibition of SARS-CoV-2 (previously 2019-nCoV) infection by a highly potent pan-coronavirus fusion inhibitor targeting its spike protein that harbors a high capacity to mediate membrane fusion. Cell Research, 2020: p. 1-13.

1. A recombinant polypeptide comprising a first portion and a second portion, the first portion comprising a resistin trimerization domain and the second portion comprising a viral surface antigen or a fragment thereof.

2. The recombinant polypeptide of claim 1, wherein the first portion comprises an amino acid sequence having at least 85% sequence identity to the full length of the amino acid sequence set forth in SEQ ID NO: 1 or SEQ ID NO: 2.

3-5. (canceled)

6. The recombinant polypeptide of claim 1, wherein the second portion comprises an amino acid sequence having at least 70% identity to the full length of the amino acid sequence set forth in SEQ ID NO: 3, SEQ ID NO: 4, or SEQ ID NO: 16.

7. The recombinant polypeptide of claim 1, comprising an amino acid sequence having a least 85% identity to the full length of the amino acid sequence set forth in SEQ ID NO: 5.

8. The recombinant polypeptide of claim 1, comprising the amino acid sequence set forth in SEQ ID NO: 5, SEQ ID NO: 6, or SEQ ID NO: 17.

9-13. (canceled)

14. A trimeric protein comprising three recombinant polypeptides, wherein each of the three recombinant polypeptides is as defined in claim 1.

15. (canceled)

16. (canceled)

17. A composition comprising the recombinant polypeptide of claim 1 and a pharmaceutically acceptable carrier.

18. The composition of claim 17, further comprising an adjuvant.

19. (canceled)

20. (canceled)

21. An expression vector for producing the recombinant polypeptide of claim 1, the expression vector comprising a nucleic acid molecule encoding the recombinant polypeptide operably linked to a promoter.

22. (canceled)

23. (canceled)

24. The expression vector of claim 21, wherein the nucleic acid molecule comprises the nucleotide sequence set forth in SEQ ID NO: 7, SEQ ID NO: 8, or SEQ ID NO: 18.

25. A method for expressing the recombinant polypeptide of claim 1, the method comprising: introducing an expression vector into a host cell, said expression vector comprising a nucleic acid molecule encoding the recombinant polypeptide operably linked to a promoter, and maintaining the cell under conditions that allow the recombinant polypeptide to be expressed.

26-28. (canceled)

29. A method of identifying whether a subject has developed an antibody response to a pathogen or to an antigen from the pathogen, the method comprising:
a. contacting serum or a serum fraction from the subject with a recombinant polypeptide as defined in claim 1, and
b. detecting binding of the recombinant polypeptide by an antibody present in the serum or the serum fraction,
wherein the second portion of the recombinant polypeptide comprises an amino acid sequence from an antigen of the pathogen.

30. The method of claim 29, wherein the pathogen is SARS-CoV-2 and the recombinant polypeptide comprises an amino acid sequence having a least 85% identity to the full length of the amino acid sequence set forth in SEQ ID NO: 5.

31. A recombinant polypeptide as defined in claim 1, wherein the recombinant polypeptide is immobilized on a surface.

32. A device comprising the recombinant polypeptide of claim 31.

33. The device of claim 32, wherein the device is a microtiter plate or a test cassette.

34. A kit comprising the recombinant polypeptide as defined in claim 1, and an antigen-binding molecule that specifically binds the recombinant polypeptide.

35. The kit of claim 34, wherein the antigen-binding molecule is an antibody or an antigen-binding fragment thereof.

36. (canceled)

37. The recombinant polypeptide of claim 2, wherein the second portion comprises an amino acid sequence having at least 70% identity to the full length of the amino acid sequence set forth in SEQ ID NO: 3, SEQ ID NO: 4, or SEQ ID NO: 16.

38. A trimeric protein comprising three recombinant polypeptides, wherein each of the three recombinant polypeptides is as defined in claim 37.

39. A method of identifying whether a subject has developed an antibody response to SARS-CoV-2 or to an antigen from SARS-CoV-2, the method comprising:
a. contacting serum or a serum fraction from the subject with a recombinant polypeptide as defined in claim 37, and
b. detecting binding of the recombinant polypeptide by an antibody present in the serum or the serum fraction.

40. A device comprising the recombinant polypeptide of claim 37 immobilized on a surface.
